(19) United States
(12) Patent Application Publication
(10) Pub. No.: US 2026/0337544 A1
(43) Pub. Date: Oct. 29, 2026

(54) PLASMA-FLUORINATED POROUS FRIT AND STAGED ANALYTICAL PROCESSING DEVICES WITH SORPTIVE, SURFACE-INTERACTION, AND STACKED CHEMISTRY MODULES FOR ENHANCED ANALYTE SEPARATION AND EXTRACTION

(71) Applicant: In-frit Extraction Technologies Inc., Palm Coast, FL (US)

(72) Inventors: Thomas Wohleb, Coeur D'Alene, ID (US); Robert Henry Wohleb, III, Eureka, CA (US); Robert Henry Wohleb, II, Palm Coast, FL (US)

(21) Appl. No.: 19/721,334

(22) Filed: Jun. 26, 2026

Related U.S. Application Data
(63) Continuation of application No. PCT/US25/27867, filed on May 6, 2025.
(60) Provisional application No. 63/716,032, filed on Nov. 4, 2024.

(51) Int. Cl. C12M 1/00; C12M 1/26; C12M 1/36; C12M 1/40; C12M 3/00

(52) U.S. Cl. CPC C12M 43/00; C12M 21/18; C12M 23/42; C12M 29/04; C12M 33/04; C12M 41/48

(57) ABSTRACT

A staged analytical processing device and method for separating, extracting, reacting, cleaning, or otherwise processing analytes from fluid samples. The device includes a housing defining a single continuous fluid path and a plurality of discrete porous processing modules disposed sequentially in the fluid path. Each porous processing module includes a porous body having interconnected pores with internal pore surfaces bearing functional chemistry so that the porous body itself functions as an active stationary phase rather than merely as a passive frit retaining loose particles. In some embodiments, a porous frit composed of sintered thermoplastic polymer particles is plasma fluorinated before application of a sorptive polymer, surface-interaction chemistry, immobilized enzyme, ion-exchange chemistry, affinity chemistry, reversed-phase chemistry, mixed-mode chemistry, or other analyte-separation chemistry. Functional staging regions between adjacent modules separate frits, prevent liquid from an upstream frit from immediately contacting downstream chemistry, and allow the liquid to be externally conditioned or port-conditioned before the next interaction. Oscillatory aspirate/dispense operation enhances mass transfer through the interconnected pore network. The architecture enables stacked chemistries such as capture, digestion, desalting, cleanup, elution, and purification to be performed within a single disposable device while reducing transfers to separate processing devices.

### CROSS REFERENCE TO RELATED APPLICATIONS

This application claims priority to U.S. Provisional Patent Application No. 63/716,032, filed Nov. 4, 2024, entitled “Porous frit coated with polymers above their glass transition temperature,” and to Patent Cooperation Treaty Application No. PCT/US 25/27867 filed May 6, 2025, entitled “Plasma Fluorinated Porous Frit With Sorptive Polymer Coating for Analyte Extraction,” each of which is incorporated herein by reference in its entirety.

### FIELD OF THE INVENTION

The present invention relates generally to sample preparation and analyte extraction devices and methods. More specifically, the invention relates to a porous frit device with a sorptive polymer coating for extracting small molecules from complex biological and aqueous matrices.

### BACKGROUND OF THE INVENTION

The preparation of biological and environmental samples for analytical detection represents one of the most persistent bottlenecks in modern laboratory workflows. Despite substantial advances in detection technology, including the widespread adoption of high-resolution liquid chromatography coupled with tandem mass spectrometry, the sample preparation steps that precede detection have remained largely unchanged in their fundamental architecture for decades. The core challenge is not the availability of chemistries capable of isolating target analytes from complex matrices. Reversed-phase sorbents, ion exchange resins, affinity ligands, and immobilized enzymes are all well-established tools. The challenge is that the workflow steps required to use these tools effectively are distributed across multiple separate consumables, multiple liquid transfers, and multiple processing stages, each of which introduces opportunity for sample loss, contamination, variability, and error. It is this architectural limitation, rather than any deficiency in the underlying chemistry, that represents the primary unresolved problem in sample preparation today.

The scale of this problem is evident across the principal segments of the analytical laboratory industry. Instrument manufacturers such as AGILENT TECHNOLOGIES, THERMO FISHER SCIENTIFIC, and WATERS CORPORATION have invested heavily in developing high-throughput mass spectrometry platforms capable of processing hundreds of samples per day. The throughput of these detection systems routinely exceeds the throughput of the sample preparation workflows that feed them. A typical clinical or research laboratory running testosterone, vitamin D metabolites, or immunosuppressive drug panels by LC-MS/MS must perform protein precipitation, solid phase extraction, and reconstitution steps before a sample is ready for injection. Each of these steps requires separate consumables, separate reagent additions, and separate liquid transfers, with the overall workflow consuming twenty to thirty minutes per sample set and requiring substantial manual or robotic intervention at each stage. The detection instrument waits. Automation companies including TECAN GROUP and HAMILTON COMPANY have developed sophisticated robotic liquid handling platforms specifically to manage this complexity, but the fundamental limitation is not the liquid handling hardware. It is the number of separate functional steps that must be performed on separate consumables and the number of times sample must be transferred between vessels before it reaches the detector.

The proteomics field illustrates the problem in its most acute form. A standard bottom-up proteomics workflow requires protein capture or precipitation from a biological matrix, enzymatic digestion with a protease such as trypsin, desalting of the resulting peptides, and chromatographic fractionation or cleanup prior to mass spectrometric analysis. These operations are currently performed using a combination of protein precipitation tubes, digestion vessels, solid phase extraction cartridges, and desalting columns, each sourced from a different product line and each requiring separate handling steps. Sample preparation companies including BIOTAGE, PHENOMENEX, and RESTEK CORPORATION supply the individual SPE consumables used in these workflows, and while each individual product is well optimized for its specific function, the architecture requires the analyst to move sample between them. Each transfer step is a source of quantitative loss. Each separate device adds dead volume. Each additional handling step adds variability. The combined effect is that bottom-up proteomics workflows are time-consuming, labor-intensive, and difficult to automate at high throughput, despite the fact that the individual chemical operations involved are individually well understood.

Conventional solid phase extraction devices, including those commercially available from the principal sample preparation suppliers, are built around a particle-retained-by-frit architecture in which a bed of particulate sorbent material is confined within a housing by one or more porous frits. The frits in these systems serve exclusively as mechanical barriers. The functional chemistry resides on or within the retained particles, and the frits themselves play no role in the analytical operation. This architecture, exemplified by products such as the OASIS SPE cartridge line from Waters Corporation, the BOND ELUT series from Agilent Technologies, and the STRATA series from Phenomenex, requires analytes to diffuse through potentially long and tortuous pathways within porous particles to reach active binding sites. The resulting diffusion limitations slow extraction kinetics, require extended processing times, and create performance dependencies on flow rate, particle size uniformity, and packing density that are difficult to control across manufacturing batches. Additionally, these devices are designed for single-function operation. A reversed-phase SPE cartridge captures analytes by hydrophobic interaction. It cannot subsequently perform ion exchange cleanup or enzymatic digestion. If the workflow requires those additional operations, separate devices must be used and sample must be transferred between them.

Prior art approaches have attempted to address workflow complexity through mixed-mode sorbent chemistries that combine two interaction mechanisms within a single particle. Products such as the OASIS MAX and OASIS MCX cartridges from Waters Corporation, the STRATA-XL-C series from Phenomenex, and the HYBRIDSPE line from Sigma-Aldrich incorporate particles bearing both hydrophobic and ionic functional groups, allowing some degree of orthogonal selectivity within a single device. While these mixed-mode sorbents reduce the number of separate devices required for certain workflows, they do not resolve the fundamental architectural limitation. The chemistries are intermixed within the same particle bed rather than spatially separated, which means that the extraction conditions applied to the sample at any given time must simultaneously satisfy the requirements of both interaction mechanisms. This constrains the range of conditions that can be applied during loading, washing, and elution, and prevents the use of conditions optimized independently for each functional step. More fundamentally, mixed-mode sorbents cannot accommodate sequential operations that are chemically incompatible with each other, such as the combination of affinity capture under alkaline conditions followed by enzymatic digestion under neutral conditions followed by reversed-phase cleanup under acidic conditions. These workflows require not only different chemistries but different solution conditions at each step, and those conditions cannot be simultaneously satisfied by a mixed particle bed.

Immobilized enzyme reactors represent a separate line of prior art development directed at integrating enzymatic processing steps into chromatographic workflows. Devices such as the IMER columns used in on-line digestion workflows and the immobilized trypsin products available from suppliers including Thermo Fisher Scientific provide enzymatic activity in a flow-through format. However, these devices are single-function reactors that perform digestion but do not capture analytes, remove matrix interferences, or purify reaction products. Using an immobilized enzyme reactor within a proteomics or metabolomics workflow still requires separate upstream capture devices and downstream cleanup devices, with sample transfer between each. The integration of enzymatic processing with chromatographic capture and purification within a single continuous flow path has not been addressed by the prior art.

Automated filter tip extraction systems, including those used in conjunction with the TECAN FREEDOM EVO and HAMILTON STAR liquid handling platforms, have extended the SPE concept to pipette tip format, enabling automated aspiration and dispensing of samples through sorbent-containing tips. Products in this category include the TIPS SPE format from various suppliers and proprietary tip-based extraction systems used in clinical sample preparation workflows. These systems reduce manual handling requirements but do not alter the fundamental single-function limitation of conventional SPE. Each tip contains a single sorbent chemistry performing a single extraction function, and multi-step workflows still require multiple tip types, multiple aspiration and dispensing cycles with different reagents, and collection and redistribution of sample between stages. The automation hardware manages the complexity of these multi-step protocols but cannot reduce the number of functional steps required, because the devices themselves are not capable of performing more than one distinct analytical operation. Disposable pipette extraction devices and related tip-based solid phase extraction systems provide useful background but retain the same fundamental particle-retained architecture. In such systems, exemplified by DPX-type pipette tips, loose or packed commercial SPE particles are positioned in a mixing chamber or bed between liquid-permeable closures, screens, barriers, or frits. The sorbent particles provide the analytical chemistry, while the frits primarily retain the particles and permit liquid flow. Although these devices may support aspirate-and-dispense mixing and may use different conventional sorbents, they do not convert the frit itself into an active stationary phase or a discrete processing module having functional chemistry on internal pore surfaces. These prior tip-based systems also do not provide a general stacked-chemistry architecture in which multiple porous processing modules, each bearing independently selected chemistry, are arranged in a single continuous fluid path with functional staging regions between modules. The absence of such staging limits the ability to perform workflows requiring chemically incompatible or temporally separated operations, such as capture under one pH condition, buffer exchange, enzymatic digestion under a second condition, and subsequent cleanup or re-capture under a third condition, within a single disposable device.

The recognition that the primary limitation of current sample preparation technology is architectural rather than chemical establishes the problem that the present invention addresses. What is needed is a device architecture that enables multiple chemically distinct and spatially separated processing operations to be performed sequentially within a single continuous flow path, without requiring sample transfer between devices, without requiring the simultaneous satisfaction of chemically incompatible conditions within a shared sorbent bed, and without the diffusion limitations inherent in particle-retained-by-frit designs. Such an architecture would collapse workflows that currently require multiple separate consumables and multiple handling steps into a single disposable device, fundamentally improving throughput, reproducibility, and automation compatibility in analytical sample preparation. The present invention provides such an architecture through a device comprising multiple discrete porous processing modules, each independently functionalized with a distinct analytical chemistry, arranged in sequence within a single continuous fluid path with functional staging regions positioned between modules to provide the chemical and temporal isolation necessary for each processing step to operate under independently optimized conditions.

### SUMMARY OF THE INVENTION

The present invention in a preferred embodiment provides a revolutionary approach to sample preparation and analyte extraction through a uniquely engineered sorptive extraction device. The invention comprises a porous frit composed of sintered plastic polymer particles that undergoes plasma fluorination treatment prior to being coated with a sorptive polymer that remains above its glass transition temperature at room temperature.

In one aspect, the invention provides a device for extracting analytes from fluid samples that can be integrated into various fluid path configurations. The device's three-dimensional porous structure enables both convective and diffusive transport of analytes into the sorptive coating under dynamic or static conditions. The porous frit has a pore size range of 10 to 200 microns and an internal surface area of approximately 0.3 square meters per gram, optimizing extraction efficiency while maintaining consistent flow characteristics.

In another aspect, the invention provides a method of manufacturing the extraction device through plasma fluorination of the frit surface using fluorine-containing gases, followed by application of a sorptive polymer coating using a solvent/anti-solvent system. The plasma treatment serves the dual purpose of reducing non-specific analyte binding while enhancing coating adhesion.

The invention further provides a simplified three-step extraction process comprising extraction, rinse, and elution steps that can be completed in under two minutes. This represents a significant improvement over traditional 20-30 minute protocols while achieving superior performance.

Key advantages of the invention include immunity to clogging from complex biological samples, elimination of emulsion formation, and prevention of analyte breakthrough. The device requires no preconditioning steps and enables effective matrix rinsing for clean extracts. The technology demonstrates broad applicability across different analyte types and matrices, successfully extracting a variety of compounds with high reproducibility.

In another aspect, an embodiment of the invention provides alternative coating chemistries that operate through surface interaction mechanisms distinct from the sorptive polymer approach. These surface interaction systems enable specific molecular recognition through targeted chemical interactions, expanding the range of analytes and applications addressable by the technology.

An embodiment of the invention further provides modular stacked configurations comprising multiple frit modules with distinct functional chemistries positioned sequentially within a single device. Void chambers or functional staging regions between modules separate adjacent frit modules, prevent liquid associated with an upstream frit from immediately contacting a downstream frit, and permit control over when that liquid next encounters downstream chemistry. In preferred pipette-tip or aspirate/dispense implementations, the staging region enables the processed liquid to be dispensed to an external vessel, such as a well of a well plate, where pH, solvent strength, buffer composition, reagent content, incubation conditions, or other process conditions may be adjusted before the liquid is aspirated back into the device and advanced to the next frit module. In ported or otherwise interfaced embodiments, reagents may additionally be introduced directly into the staging region. Sealing arrangements prevent bypass flow to ensure complete sample processing through the selected modules.

In yet another aspect, an embodiment of the invention provides extensive manufacturing specifications including plasma parameter optimization based on frit material and coating type, quality control methods for coating uniformity verification, and scale-up considerations for commercial production consistency.

The technology demonstrates particular advantages in providing shorter diffusion paths compared to conventional particle-based solid phase extraction systems. The frit-as-stationary-phase architecture eliminates the need for particulate sorbents retained by frits, instead providing functional chemistry directly on the frit pore surfaces where analytes can interact without long diffusion paths through porous particles. The invention is not limited to a sorptive polymer embodiment. In broader embodiments, the invention provides a platform in which porous frit bodies or other porous processing modules function as active stationary phases bearing any of a plurality of analyte-separation or analyte-processing chemistries on internal pore surfaces. Such chemistries may include, by way of non-limiting example, reversed-phase chemistries, ion-exchange chemistries, mixed-mode chemistries, affinity ligands, immobilized metal affinity chemistries, immobilized enzymes, derivatization reagents, desalting chemistries, molecular-weight selection materials, hydrophilic interaction chemistries, hydrophobic interaction chemistries, sorptive partitioning polymers, and multi-modal combinations thereof.

In still further embodiments, the porous processing module is substantially free of a loose particulate sorbent bed retained between passive frits. Instead, the porous body itself provides both structural flow-through support and analytical function. This distinction is significant because the analyte need not diffuse into retained particles or through a packed bed to reach active sites; rather, the analyte encounters functional chemistry directly on accessible internal pore surfaces of the porous body during convective and diffusive transport through the module.

### BRIEF DESCRIPTION OF THE FIGURES

FIG. 1 is a cross-sectional view illustrating the internal structure of a sorptive coated frit.

FIG. 2 depicts a cross-sectional view of a pipette tip, showing the placement of the coated frit within the tip body.

FIG. 3 presents a cross-sectional view of a cartridge system, such as an SPE cartridge, incorporating the coated frit.

FIG. 4 shows a cross-sectional view of a spin column device featuring the coated frit at its bottom exit.

FIG. 5a and FIG. 5b provide schematic top and cross-sectional views, respectively, of a microfluidic device with an integrated coated frit.

FIG. 6 displays an exploded cross-sectional view of a syringe filter assembly, illustrating the position of the coated frit within the housing.

FIG. 7 is a comparative diagram illustrates the fundamental architectural departure from conventional particle-retained systems showing the inventive frit-as-stationary-phase approach versus prior art particle-based architectures in accordance with an embodiment of the invention.

FIG. 8: A process flow diagram shows the plasma fluorination and dual-purpose surface modification process demonstrating the transformation from native polymer surface to fluorinated surface optimized for coating adhesion in accordance with an embodiment of the invention.

FIG. 9: An exploded view illustrates the modular stacked configuration with void chambers or functional staging regions showing sequential frit modules for capture, reaction, and purification within a single device housing in accordance with an embodiment of the invention.

FIG. 10: A three-panel time sequence demonstrates the oscillatory flow dynamics and enhanced mass transfer showing aspiration, dispensing, and equilibrium achievement phases for superior extraction performance in accordance with an embodiment of the invention.

### DETAILED DESCRIPTION

In a preferred embodiment of the invention, a high surface area, low dead volume porous frit is composed of sintered thermoplastic polymer particles. The frit is generally a porous structure formed from sintered thermoplastic particles that create an interconnected porous network capable of supporting surface functionalization throughout the internal pore structure. Preferred materials include polyolefins such as ultra-high molecular weight polyethylene (UHMWPE), high-density polyethylene (HDPE), and polypropylene, although other thermoplastics suitable for sintering into stable porous bodies may also be used. The frit dimensions can vary based on the intended fluid path configuration, ranging from approximately 0.5 mm×1 mm for small volume applications like 20 μl pipette tips to larger dimensions such as 4.5 mm×6.5 mm for standard configurations. The frit may also be conical or otherwise conform to non-standard tubular shapes to match specific flow path requirements. The frit material in this embodiment is formed from sintered polyolefin particles and is plasma treated to modify the internal pore surfaces prior to application of the stationary phase coating, the treatment being conducted in the presence of fluorine-containing plasma species to improve coating adhesion and reduce non-specific analyte interactions with the underlying polymer substrate.

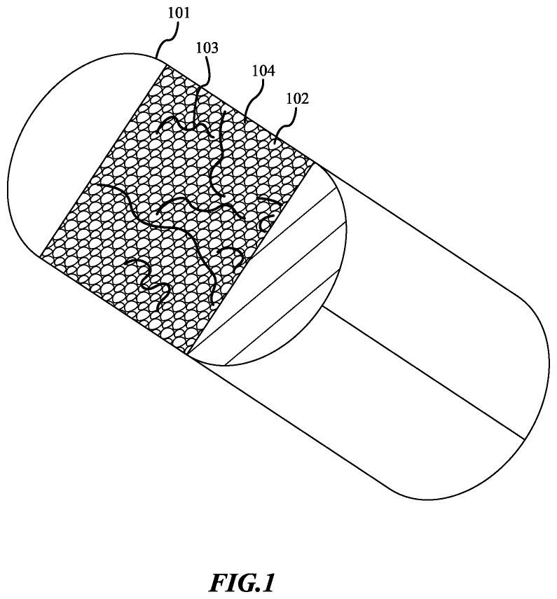

An embodiment of the invention is illustrated in FIG. 1, which shows a cross-sectional view of the internal structure of a sorptive coated frit. The frit body (101) comprises sintered plastic polymer particles that have been fused together to create a unified porous structure with consistent porosity and mechanical integrity. The polyethylene particles (102) represent the individual sintered particles that form the three-dimensional porous matrix of the frit, wherein the particles are composed of high-density polyethylene or polypropylene materials as described in the preferred embodiments. The sorptive coating (103) is applied uniformly across the internal surface area of the frit at approximately 3 microns thickness, functioning as an immobilized liquid that enables analytes to dissolve into the polymer phase rather than merely adsorbing to surfaces. Representative pores (104) illustrate the interconnected void spaces throughout the frit structure, with pore sizes ranging from 10 to 200 microns that enable both convective and diffusive transport of analytes into the sorptive coating under dynamic or static conditions. The porous structure maintains an internal surface area of approximately 0.3 square meters per gram while providing the three-dimensional geometry necessary for optimal mass transfer characteristics.

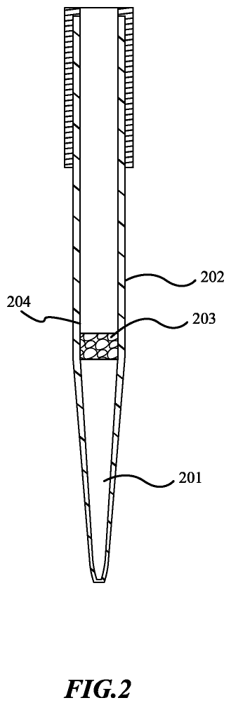

An embodiment of the invention is depicted in FIG. 2, showing a cross-sectional view of a pipette tip configuration incorporating the coated frit. The conus region (201) represents the tapered portion of the pipette tip where the frit is positioned, providing the appropriate geometric constraints for secure frit insertion and proper flow path characteristics. The pipette main body (202) comprises the primary cylindrical structure of the pipette tip that connects to standard liquid handling systems, maintaining compatibility with both manual and automated pipetting operations. The coated frit (203) is positioned within the conus region and represents the porous frit that has undergone plasma fluorination treatment followed by sorptive polymer coating application, enabling the three-step extraction, rinse, and elution workflow described in the preferred embodiments. The tip transition point (204) indicates the specific location where the frit is pressed firmly into position, creating a compression seal against the internal walls of the pipette tip to prevent bypass flow around the edges while maintaining proper flow path geometry for optimal analyte-stationary phase interaction.

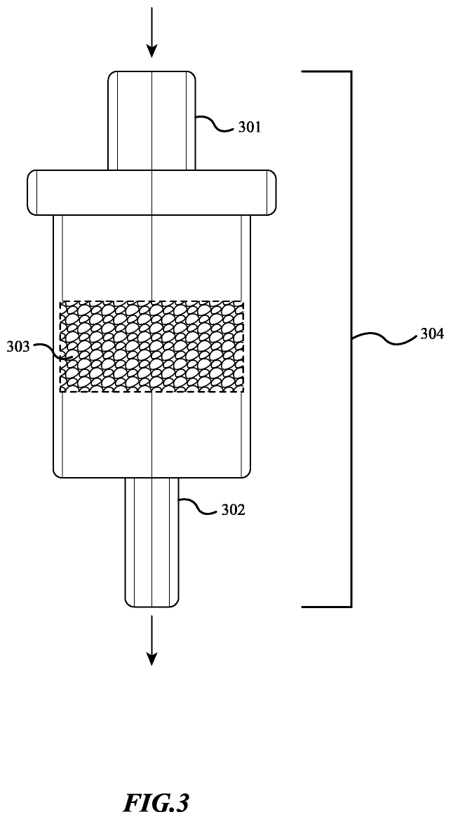

An embodiment of the invention is shown in FIG. 3, presenting a cross-sectional view of a cartridge system such as a solid-phase extraction (SPE) cartridge incorporating the coated frit. The female luer fitting (301) provides a standardized connection interface that enables secure attachment to syringes, pumps, or other fluid delivery systems commonly used in analytical laboratories. The male luer fitting (302) serves as the outlet connection, allowing for controlled sample collection and maintaining leak-tight seals under typical operating pressures encountered in extraction workflows. The frit (303) represents the plasma-fluorinated porous frit with sorptive polymer coating positioned within the cartridge housing, wherein the frit functions as the stationary phase without requiring particulate sorbent materials retained by mechanical barriers. The dimensional specifications of the cartridge system (304) indicate the overall geometric parameters optimized for integration with standard laboratory equipment while providing appropriate internal volume ratios between the sample and the sorptive polymer coating to maximize extraction efficiency and phase ratios.

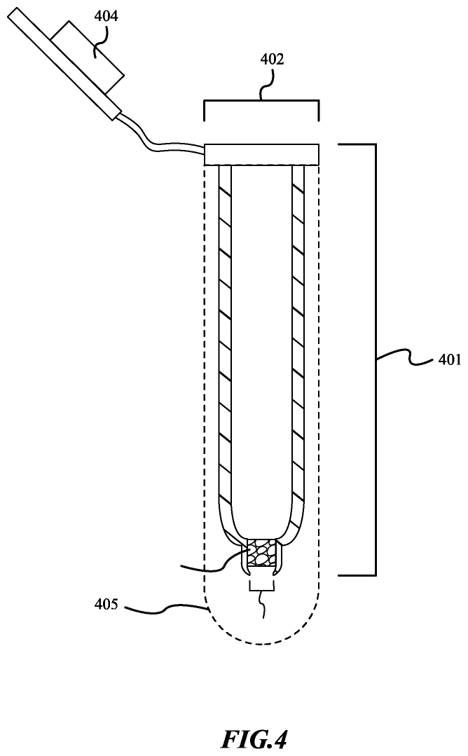

An embodiment of the invention is illustrated in FIG. 4, showing a cross-sectional view of a spin column device featuring the coated frit positioned at the bottom exit for centrifugation-based sample processing. The height (401) represents the overall vertical dimension of the spin column designed to fit within standard centrifuge rotors while providing sufficient volume for sample processing and mechanical integrity under centrifugal forces ranging from 1,000 to 16,000×g. The top diameter (402) indicates the opening dimensions designed for sample introduction and compatibility with standard laboratory pipettes and dispensing systems. The frit (404) is positioned at the exit point of the spin column and comprises the plasma-fluorinated porous structure with sorptive polymer coating that enables analyte extraction during centrifugal processing. The collection tube (405) provides the receptacle for collecting processed samples and eluates, with dimensional specifications that ensure proper fit within centrifuge configurations while maintaining appropriate volume capacity for the analytical workflow.

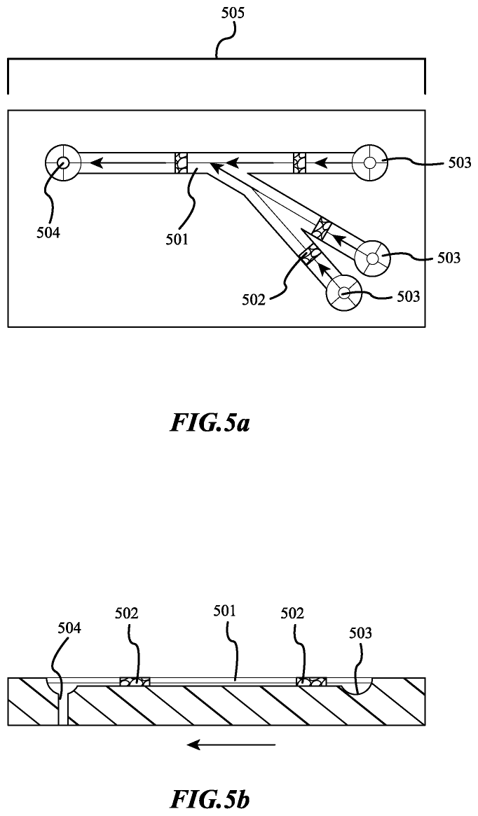

An embodiment of the invention is depicted in FIG. 5a, showing a schematic top view of a microfluidic device with an integrated coated frit for lab-on-chip applications. The microfluidic channel (501) represents the flow path designed with dimensional tolerances compatible with standard microfluidic channel geometries and flow rate specifications appropriate for microfluidic analytical applications. The frit (502) is integrated within the microfluidic channel and comprises the plasma-fluorinated porous structure with sorptive polymer coating that maintains extraction capabilities while fitting within the constrained dimensions of microfluidic systems. The inlet ports (503) provide multiple entry points for sample introduction, reagent addition, or buffer delivery, enabling complex analytical workflows within the integrated microfluidic platform. The outlet ports (504) allow for controlled sample collection and waste removal, with the port configuration designed to accommodate various analytical protocols including the three-step extraction, rinse, and elution process. The scale bar (505) provides dimensional reference indicating the microscale dimensions typical of microfluidic devices while demonstrating the ability to integrate the sorptive extraction technology at reduced scales.

An embodiment of the invention is shown in FIG. 5b, presenting a cross-sectional view of the microfluidic device that reveals the internal structure and flow path configuration. The frit (502) is positioned within the microfluidic channel cross-section, demonstrating how the three-dimensional porous structure is integrated within the planar microfluidic architecture while maintaining the ability to provide both convective and diffusive transport of analytes into the sorptive coating. The microfluidic channel (501) cross-section shows the internal geometry optimized for laminar flow characteristics while accommodating the porous frit structure. The inlet ports (503) and outlet ports (504) are shown in cross-section, illustrating the fluidic connections that enable sample processing through the integrated extraction system. The scale bar (505) maintains the dimensional reference for the microscale implementation, demonstrating how the sorptive extraction principles can be applied to microfluidic analytical platforms while preserving the fundamental extraction mechanisms described in the preferred embodiments.

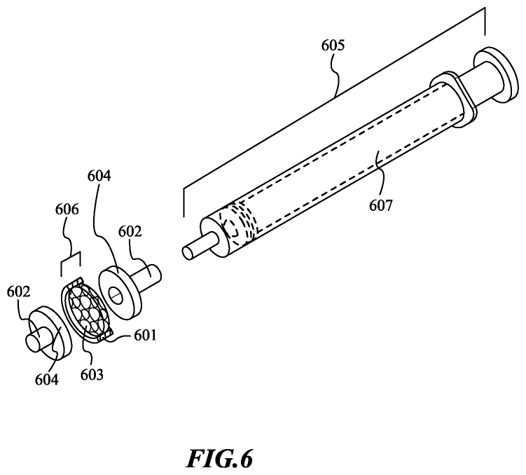

An embodiment of the invention is depicted in FIG. 6, showing an exploded cross-sectional view of a syringe filter assembly that illustrates the position of the coated frit within a filter housing configuration. The filter housing (601) provides the structural enclosure that contains the frit while maintaining mechanical integrity and providing appropriate flow path geometry for syringe-driven sample processing. The luer lock fittings (602) enable secure connections to standard laboratory syringes and ensure leak-tight seals during manual or automated sample processing operations. The frit (603) represents the plasma-fluorinated porous structure with sorptive polymer coating positioned within the housing, where it functions as the stationary phase for analyte extraction from samples introduced through syringe pressure. The filter supports (604) provide mechanical support for the frit while maintaining open flow paths and preventing deformation under applied pressure during sample processing. The dimension bar for the syringe (605) and the dimension bar for the filter housing (606) provide scale references for the overall assembly dimensions, while the syringe (607) illustrates the typical sample delivery mechanism used with this configuration, enabling precise sample volume control and consistent extraction performance.

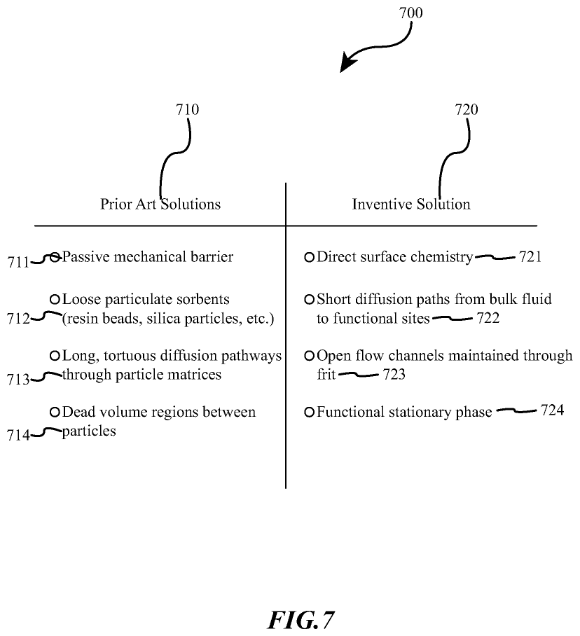

An embodiment of the invention is illustrated in FIG. 7, which provides a comparative diagram distinguishing prior art solutions (700, 710-714) from the inventive approach (720-724). The prior art solutions section (700) demonstrates conventional particle-retained-by-frit systems where the passive mechanical barrier (711) serves merely to contain loose particulate sorbents (712) such as resin beads and silica particles. These conventional systems create long, tortuous diffusion pathways (713) through particle matrices that slow extraction kinetics and may result in incomplete analyte recovery, while dead volume regions between particles (714) contribute to sample carryover and reduced analytical precision. In contrast, the inventive solution (720) demonstrates the revolutionary frit-as-stationary-phase architecture where direct surface chemistry (721) resides on the internal pore surfaces of the frit itself. The inventive approach provides short diffusion paths from bulk fluid to functional sites (722), enabling rapid mass transfer and efficient extraction performance. Open flow channels maintained through the frit (723) ensure consistent flow characteristics while the functional stationary phase (724) eliminates the need for particulate sorbents entirely, providing functional chemistry directly accessible to analytes without the diffusion limitations characteristic of particle-based systems.

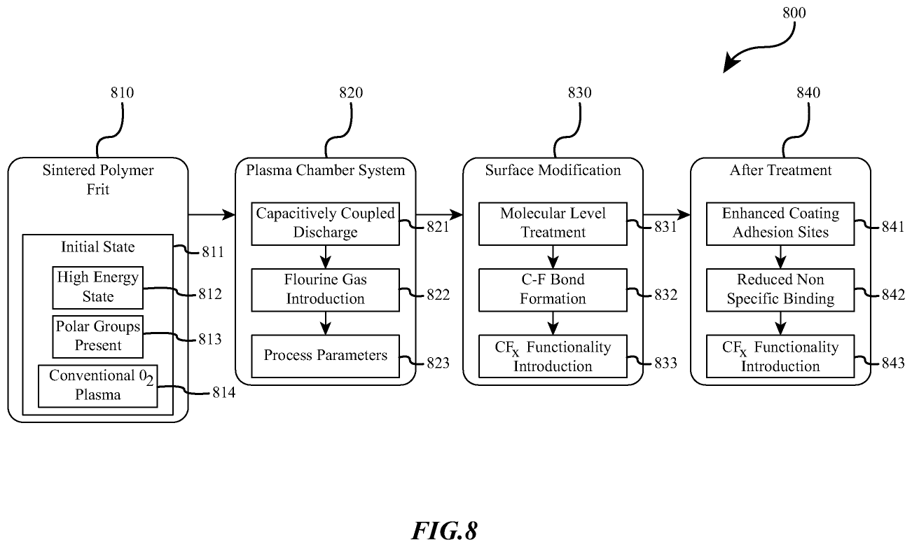

An embodiment of the invention is shown in FIG. 8, illustrating the plasma fluorination and dual-purpose surface modification process (800) that enables optimal coating adhesion while reducing non-specific analyte binding. The sintered polymer frit (810) begins in its initial state (811) with high energy surface characteristics (812) and polar groups present (813) from conventional surface treatments. The plasma chamber system (820) utilizes capacitively coupled discharge (821) with fluorine gas introduction (822) and controlled process parameters (823) to modify the frit surface. The surface modification stage (830) creates molecular level treatment (831) through CFx functionality introduction (832-833), fundamentally altering the surface chemistry compared to conventional O2 plasma treatments. The after treatment result (840) demonstrates enhanced coating adhesion sites (841), reduced non-specific binding characteristics (842), and CFx functionality introduction (843) that creates optimal surface conditions for subsequent sorptive polymer coating application. This dual-purpose plasma fluorination process addresses both analytical performance requirements and manufacturing consistency by creating a surface that is chemically compatible with low-surface-energy polymers while maintaining reduced background interactions essential for analytical applications.

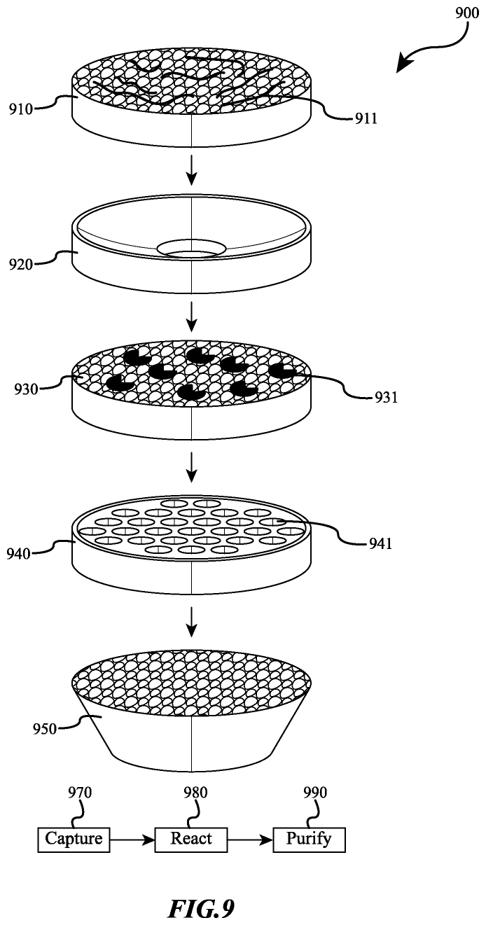

An embodiment of the invention is depicted in FIG. 9, showing a modular stacked configuration (900) that enables complex multi-step analytical workflows within a single disposable device. The capture module (910) contains a frit with affinity ligands coating (911) designed for analyte capture from complex biological matrices, providing the initial selectivity for target compounds. Void chamber 1 (920) provides an isolation and staging space between modules, thereby separating adjacent frits, preventing liquid from one frit from immediately contacting the next frit, and allowing the timing of contact with downstream chemistry to be controlled. In a preferred workflow, liquid exiting or released from an upstream module may be dispensed from the device into an external well plate or other external vessel, externally conditioned by adjustment of pH, solvent composition, ionic strength, buffer composition, reagent content, incubation time, or temperature, and then aspirated back through the device before contacting the next frit module. In embodiments having a port, septum, side channel, valve, or other fluidic interface communicating with the void chamber, reagent addition or condition adjustment may be performed directly in the void chamber. The enzymatic reaction frit (930) contains immobilized enzymes (931) such as trypsin or Lys-C for biochemical transformations including proteolytic digestion, with the frit architecture providing optimal surface area for enzyme immobilization. Void chamber 2 (940) provides additional isolation, staging, buffer-exchange, pH-adjustment, or solvent-strength-adjustment capacity before the processed liquid contacts the purification frit (950), which performs final cleanup using chromatographic phases such as reversed-phase or ion exchange chemistry. The sequential workflow demonstrates capture (970), reaction (980), and purification (990) steps that can be completed within the single integrated device, eliminating or reducing sample transfer operations between different disposable processing devices and reducing contamination risks while enabling sophisticated analytical workflows not achievable with conventional single-function extraction devices.

As used herein, a functional staging region refers to a void volume disposed within the single continuous fluid path between adjacent discrete porous processing modules. The functional staging region is deliberately incorporated into the device architecture primarily to separate adjacent frit modules, prevent liquid associated with an upstream frit module from immediately contacting a downstream frit module, and allow the user or automated system to control when the processed liquid next encounters downstream chemistry. The functional staging region is distinguished from a mere gap or incidental dead volume by its deliberate use as an isolation and timing region within a sequential analytical workflow. First, hydrodynamic buffering is provided because the functional staging region spatially separates adjacent discrete porous processing modules such that upstream processing conditions, reagents, matrix components, or released analytes do not prematurely contact downstream functional chemistries before solution conditions are appropriately adjusted. Second, chemical isolation is provided because the functional staging region separates chemically incompatible environments maintained within adjacent discrete porous processing modules, for example separating an acidic ion exchange environment from a neutral-pH enzymatic digestion environment such that the chemical conditions optimized for each module do not interfere with one another. Third, external conditioning is enabled because liquid occupying or passing through the functional staging region may be dispensed from the device to an external vessel, such as a well plate, where the composition, pH, ionic strength, solvent strength, reagent content, incubation conditions, temperature, or other chemical characteristics of the processed fluid can be modified, adjusted, or equilibrated before the liquid is aspirated back into the device and contacted with a downstream discrete porous processing module. Fourth, in ported embodiments, buffer exchange, reagent introduction, pH adjustment, solvent-strength adjustment, or reaction staging may occur directly in the functional staging region through a port, septum, side channel, valve, reservoir, or other fluidic interface communicating with the staging region. Fifth, reactions can occur in the functional staging region when the relevant reaction conditions have been set externally before re-aspiration or when the device includes such a ported or interfaced configuration for introducing reagents or adjusting conditions directly. In oscillatory aspirate and dispense operation, the functional staging region may further promote diffusion-driven mixing and equilibration of the processed fluid before it enters the next discrete porous processing module. The volume of each functional staging region may be independently selected based on the requirements of the intended analytical workflow, with volumes ranging from approximately 5 microliters for rapid isolation or mixing applications to approximately 200 microliters for extended incubation or multi-step conditioning protocols. As used herein, a discrete porous processing module is distinguished from a passive frit, screen, filter, or liquid-permeable closure by the presence of functional chemistry on internal pore surfaces of the porous body. A passive frit may hold particles in place, filter particulates, or define a mechanical boundary. By contrast, the discrete porous processing modules described herein perform analytical work on the sample, including capture, extraction, partitioning, binding, reaction, digestion, cleanup, desalting, derivatization, or separation of analytes or interfering matrix components. In certain embodiments, the device may omit loose SPE particles entirely. In other embodiments, particulate materials may be present as auxiliary materials, but the analytical architecture remains defined by one or more functionalized porous bodies that themselves bear the operative chemistry on internal pore surfaces. The device therefore differs from conventional particle-retained-by-frit systems even when both types of devices are housed in a pipette tip, cartridge, spin column, well plate, or microfluidic flow path.

In embodiments of the invention, the modular architecture of sequentially disposed discrete porous processing modules with interposed functional staging regions enables a general workflow paradigm characterized as capture, external conditioning or transformation, and re-capture. In this paradigm, a first discrete porous processing module captures target analytes from a complex sample matrix using a first functional chemistry under conditions that favor selective analyte retention while allowing interfering matrix components to pass through or be removed by rinsing. The captured analytes or a portion thereof are then eluted or released from the first module and transferred through, or temporarily isolated by, a functional staging region. In preferred aspirate/dispense operation, the liquid is dispensed from the device into a well plate or other external vessel, where pH, ionic strength, solvent strength, buffer composition, reagent content, temperature, incubation time, or other process conditions are adjusted before the liquid is aspirated back into the device and advanced to the next processing module. In ported embodiments, analogous condition adjustment or reagent introduction may occur directly through a port or other interface communicating with the staging region. A second discrete porous processing module then performs a transformation on the transferred analytes using a second functional chemistry different from the first, wherein the transformation may comprise an enzymatic reaction such as proteolytic digestion or deglycosylation, a chemical derivatization reaction, a reduction and alkylation of disulfide bonds, or any other chemical or biochemical conversion appropriate to the analytical workflow. The transformation products are then contacted with the first discrete porous processing module, a third discrete porous processing module, or both, for re-capture, cleanup, desalting, or further fractionation prior to final elution and analytical detection. This capture, conditioning or transformation, and re-capture paradigm is not limited to any particular chemistry family and is applicable across diverse analytical workflows. In a proteomics workflow, proteins are captured by ion exchange or affinity chemistry, externally conditioned to digestion-compatible pH or solvent conditions, transformed by enzymatic digestion, and the resulting peptides are re-captured for desalting and cleanup before mass spectrometric analysis. In a metabolomics workflow, metabolites are captured by reversed-phase or mixed-mode chemistry, chemically or enzymatically deconjugated to release phase II metabolite conjugates after appropriate external or ported condition adjustment, and re-captured under modified conditions for enrichment and concentration. In a phosphoproteomics workflow, phosphopeptides are captured by immobilized metal affinity chemistry, subjected to enzymatic dephosphorylation or other modification if desired, and re-captured or fractionated on a downstream module. In a therapeutic drug monitoring workflow, drugs and active metabolites are captured from a biological matrix by affinity or adsorptive chemistry, the captured compounds are released and subjected to chemical or enzymatic conversion to analytical derivatives if required, and the derivatives are re-captured on a purification module for clean elution into the analytical instrument. The common feature of all such workflows within the inventive device is that each sequential processing operation occurs at a distinct spatial location within the single continuous fluid path, with functional staging regions providing the necessary chemical and temporal separation between operations, while external conditioning or ported conditioning supplies the chemical changes needed before the liquid encounters the next chemistry.

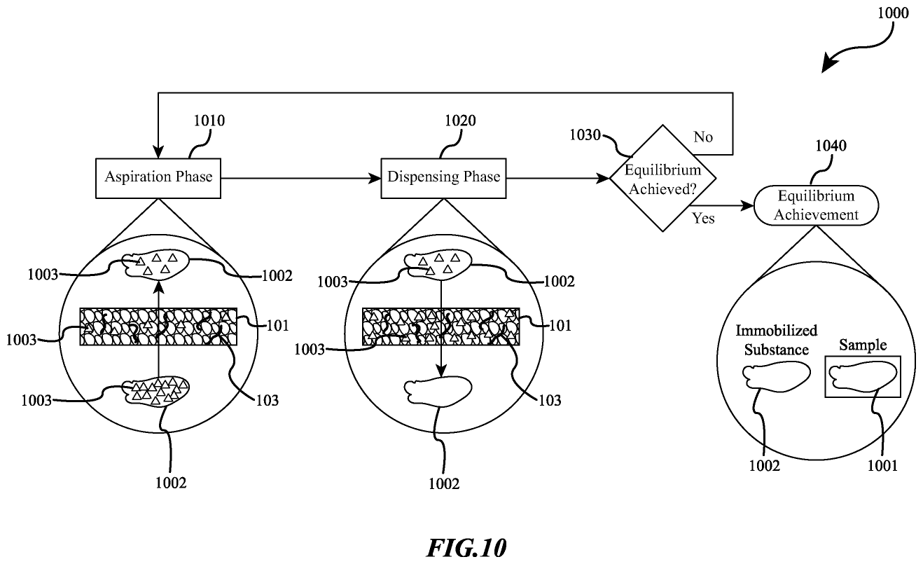

An embodiment of the invention is illustrated in FIG. 10, showing the oscillatory flow dynamics system (1000) that provides enhanced mass transfer compared to conventional single-pass flow-through methods. The aspiration phase (1010) demonstrates sample being drawn through the frit, where analytes begin partitioning into the sorptive coating applied to the internal pore surfaces. The dispensing phase (1020) shows sample being expelled, creating enhanced mixing within the pore structure that promotes convective transport effects exceeding those achievable with laminar flow systems. The equilibrium achievement determination (1030) represents the decision point where extraction completeness is evaluated, with the system proceeding to equilibrium achievement (1040) when optimal analyte-stationary phase interaction is obtained. The sample (1001) interacts with the immobilized substance (1002) representing the sorptive polymer coating, while analytes (1003) dissolve into the three-dimensional polymer matrix rather than merely adsorbing to surfaces. This oscillatory operation enables the same sample volume to interact repeatedly with fresh regions of the sorptive coating, eliminating concentration gradients and uneven extraction patterns characteristic of single-pass flow systems while achieving extraction equilibrium in seconds rather than the minutes typically required by conventional approaches.

In an example of the invention, the pore size of the frit is 40 micron with an internal surface area of 0.3 square meters per gram. The sorptive coating is approximately 3 microns thick and covers the entirety of the internal surface area of the frit.

In an embodiment, the coated frit is inserted into the conus of a 1mL pipette tip and pressed into the area just prior to the tip. The sample preparation process in this embodiment consists of three steps: extraction, rinse, and elution. The extraction is accomplished by aspirating and dispensing a sample three times over a period of 50 seconds. The rinse step is accomplished by aspirating and dispensing a rinse solution once over a period of 20 seconds. The elution step is accomplished by aspirating and dispensing an elution solution three times over a period of 50 seconds.

In another embodiment of the invention, the sorptive-coated frit may be integrated into various fluid path configurations, including cartridge systems, spin columns, microfluidic devices, and continuous flow-through assemblies. For example, the frit may be inserted into standard solid-phase extraction (SPE) cartridges or adapted for use in flow-through well plate formats. These configurations enable modular integration of the sorptive frit into both manual and automated sample processing workflows.

In a preferred embodiment, the sorptive polymer coating is an elastomeric polymer above its glass transition temperature that exhibits the extractive characteristics of a liquid while maintaining complete physical homology. Small molecules are soluble in the extracting phase, allowing partitioning to occur when an aqueous matrix is directly exposed to the polymer. The extracting elastomer is completely immiscible in aqueous sample matrices such as serum, plasma, and urine, which ensures a clean back-extraction when simple preventative measures like rinsing are taken.

An example implementation demonstrates the extraction of testosterone from human serum using a pipette tip containing the sorptive-coated frit. The process consists of three distinct steps: first, aspirating and dispensing the sample mix three times over a 50-second period; second, performing a single aspiration and dispense cycle with wash solution over 20 seconds; and third, eluting with solvent using three aspiration/dispense cycles over 50 seconds. This method achieves a lower limit of quantitation of 0.04 ng/ml, with excellent linearity and reproducibility.

In another example, the invention demonstrates extraction of THC metabolites from urine samples, achieving excellent reproducibility with a coefficient of variation of 5.6% and linearity greater than 0.998. Compared to conventional dilute-and-shoot methods, this approach shows reduced matrix effects and improved analyte recovery.

In a preferred embodiment of the invention, the porous frit is composed of sintered thermoplastic polymer particles that form a stable interconnected porous structure capable of supporting surface functionalization throughout the internal pore network. Preferred thermoplastic materials include polyolefins such as ultra-high molecular weight polyethylene (UHMWPE), high-density polyethylene (HDPE), and polypropylene, although other thermoplastic polymers suitable for sintering into stable porous bodies may also be used. These polyolefin materials are members of a broader class of polymers produced from simple olefins (alkenes) such as ethylene, propylene, butene, and their copolymers. Throughout this application, the terms “polyolefin” and “polyethylene/polypropylene” may be used interchangeably, with polyolefin serving as the broader polymer classification. The important functional characteristics of the frit material are that it can be sintered into a stable interconnected porous structure, can be surface modified throughout the pore network by plasma treatment, can support uniform internal coating or functionalization, and can maintain the mechanical properties needed for sealing within housings such as pipette tips, cartridges, and tubes. The frit is formed by sintering or fusing together small particles of these thermoplastic materials to create a single unified structure with a consistent porosity profile and mechanical integrity suitable for the intended analytical applications.

In an example embodiment, the frit is formed from sintered polyethylene particles and has dimensions of approximately 4.5 mm×6.5 mm. The sintered plastic polymer construction creates an inherent porosity within the frit structure that enables significantly greater surface area compared to non-porous, fixed coatings.

The sintered thermoplastic polymer composition provides critical advantages for the invention in accordance with a preferred embodiment. Polyolefin materials, such as polyethylene and polypropylene, are inherently chemically inert, minimizing non-specific analyte interactions. However, this inertness also makes direct coating adhesion challenging. Standard oxygen plasma treatments can introduce polar hydroxyl groups onto the surface, increasing hydrophilicity but also creating active sites that may promote undesired analyte binding. Moreover, oxygen plasma alone does not effectively modify the internal surfaces of the porous frit structure, and the treatment tends to decay relatively quickly. In contrast, plasma surface modification in the presence of fluorine-containing plasma species penetrates much more effectively throughout the porous frit structure and remains stable for extended periods prior to coating. This treatment modifies the frit surface to enhance polymer adhesion while preserving the material's low surface energy, thereby reducing non-specific interactions. The fluorine-containing plasma species may be generated from fluorinated gases introduced directly into the plasma chamber, from fluorinated polymers exposed to the plasma environment, or from other fluorine-containing compounds, with the important functional requirement being that the reactive species reach the internal pore surfaces of the porous frit. This selective surface modification enables the formation of a uniform stationary phase coating while maintaining the base polymer's desirable chemical inertness.

In another embodiment, the frit is composed of sintered high-density polyethylene particles that are plasma treated to be wettable by the polymer coating that will be subsequently applied. The sintered construction creates a three-dimensional structure with interconnected pores throughout the frit body, enabling both convective and diffusive transport of analytes.

The porous frit composition is based on sintered thermoplastic polymers, particularly sintered polyolefins, because these materials provide the specific pore structure, surface modification compatibility, and mechanical behavior needed for the device. Unlike glass-based substrates, the sintered thermoplastic frit can be effectively modified throughout its internal pore structure through plasma treatment, and the thermoplastic composition allows the frit to maintain its structural integrity while providing an appropriate substrate for the stationary phase coating. The thermoplastic nature of the material also enables the frit to form compression seals against the internal walls of device housings such as pipette tips and cartridges, preventing bypass flow during operation.

In a preferred embodiment of the invention, the porous frit has a pore size range of 10 to 200 microns, which enables optimal flow characteristics and extraction efficiency.

In an example embodiment, the frit is constructed with a pore size of 40 micron, which provides an internal surface area of 0.3 square meters per gram. This specific pore size allows for efficient convective and diffusive transport of analytes into the sorptive coating under both dynamic and static conditions.

The selected pore size range is critical for optimizing extraction performance in accordance with various embodiments. Sufficient porosity ensures a high surface area for sorptive polymer coating while maintaining low dead volume, which improves the phase ratio between the polymer phase and the fluid sample and enhances mass transfer rates. Excessively small pores or thick polymer coatings can hinder analyte diffusion into the sorptive phase, slowing extraction kinetics and reducing sensitivity. Maintaining an appropriate pore structure thus enables rapid analyte partitioning into the coating while minimizing fluidic resistance and non-productive sample volume.

In another embodiment, the pore size range enables reproducible, conformal internal coating throughout the frit geometry. The porous structure with defined pore sizes allows for effective mass transfer, as the rate of diffusion of an analyte within the sample, across the interface, or within the immobilized liquid is directly proportional to the absolute temperature and inversely proportional to the square root of the molecular weight.

The 10-200 micron pore size range also facilitates continuous flow-through operation while maintaining structural integrity. This range has been optimized based upon considerations of coating thickness, dead volume, and sample volume such that extraction time, efficiency, and phase ratio are maximized. The pore structure enables both convective mass transport during analyte extraction and effective rinsing of matrix interference components.

In a preferred embodiment of the invention, the porous frit has an internal surface area of 0.3 square meters per gram, which provides sufficient surface area for effective analyte extraction while maintaining optimal phase ratios.

The internal surface area specifications in accordance with a preferred embodiment are critical to achieving efficient analyte extraction. In one embodiment, the available surface area is optimized relative to the polymer coating thickness to maintain an effective phase ratio and to promote rapid diffusion of analytes into the sorptive layer. If surface area is insufficient relative to coating thickness, the polymer phase can become saturated at the surface, requiring analytes to diffuse deeper into the coating to reach available sorptive sites. This increases diffusion pathlengths, slows extraction kinetics, and reduces overall analytical performance. Therefore, balancing surface area and coating thickness is essential for maximizing extraction speed and capacity.

In another embodiment, the internal surface area specifications are designed to provide the same amount of polymer but with more surface area to make the functioning more efficient. The very low dead volume and high surface area are critically important in the context of the invention because of phase ratio and rate of diffusion through the polymer.

The internal surface area specifications work in conjunction with the three-dimensional geometry of the frit to enable convective mass transport during analyte extraction. This combination of specified internal surface area and geometric structure allows for both convective and diffusive transport of analytes into the sorptive coating under dynamic or static conditions.

In a preferred embodiment, the internal surface area specifications are optimized based upon considerations of coating thickness, dead volume, and sample volume such that extraction time, efficiency, and phase ratio are maximized. The specified surface area enables reproducible, conformal internal coating throughout the frit geometry while maintaining sufficient capacity for analyte extraction. An important performance characteristic of the frit architecture is the very short diffusion path between the mobile phase and the stationary phase located on the internal pore surfaces of the frit. In conventional chromatographic materials such as packed particle beds, analytes must diffuse through relatively large particles in order to reach the stationary phase, which can slow mass transfer and interaction kinetics. In contrast, the stationary phase in the present system resides on the internal pore surfaces of a porous frit. Because the pores are directly accessible to flowing liquid, analytes can rapidly diffuse to the stationary phase located on the pore walls. This geometry results in very short diffusion distances between the bulk solution and the stationary phase, enabling analyte interaction with the stationary phase to occur rapidly even during relatively short aspirate/dispense cycles or oscillatory flow conditions.

In a preferred embodiment of the invention, the three-dimensional geometry of the porous frit enables both convective and diffusive transport of analytes into the sorptive coating under dynamic or static conditions.

The three-dimensional structure is created through the sintering process which fuses the polymer particles together to form interconnected pores throughout the frit body. This geometry is critical because it allows for convective mass transport during analyte extraction while maintaining a high surface area relative to flat coatings.

In an embodiment, the three-dimensional geometry works in conjunction with the specified pore size range and internal surface area to optimize mass transfer. The overall kinetics of extraction are controlled by the characteristics of the device and coating that affect mass transfer. Mass transfer occurs through the net movement of analytes from the sample to the immobilized liquid, accomplished by the diffusion process within the sample, between the sample and immobilized liquid, and within the immobilized liquid until equilibrium is reached.

The three-dimensional structure provides several key advantages. In a preferred embodiment, the geometry enables rapid diffusion in the immobilized liquid due to small diffusion distances relative to the sample. Changes in surface area have a profound effect on kinetics, as increasing the surface area of the immobilized liquid increases the rate of diffusion across the interface directly proportional to the surface area. The concomitant reduction in diffusion distance within the sample therefore reduces the time to equilibrium.

In another embodiment, the three-dimensional geometry facilitates flow-through operation while maintaining structural integrity. The structure allows for effective rinsing of matrix interference components while providing sufficient surface area for the sorptive polymer coating. This geometric configuration has been optimized based upon considerations of coating thickness, dead volume, and sample volume such that extraction time, efficiency, and phase ratio are maximized.

In a preferred embodiment of the invention, the surface treatment process comprises plasma surface modification of the porous frit in the presence of fluorine-containing plasma species. The plasma treatment modifies the internal pore surfaces of the sintered thermoplastic frit to enable proper wetting and adhesion of the subsequent stationary phase coating while reducing non-specific analyte binding to the underlying polymer substrate.

The fluorine-containing reactive species within the plasma environment may be generated through several approaches. In one embodiment, fluorine-containing gases are introduced directly into the plasma chamber, including but not limited to tetrafluoromethane (CF₄), sulfur hexafluoride (SF₆), hexafluoroethane (C₂F₆), perfluoropropane (C₃F₈), hexafluoropropylene (C₃F₆), octafluorocyclobutane (C₄F₈), nitrogen trifluoride (NF₃), difluoromethane (CH₂F₂), trifluoromethane (CHF₃), and other fluorine-containing compounds. These gases may be used individually or in combination to achieve the desired surface modification effects.

In another embodiment, fluorine-containing plasma species are generated by exposing a fluorinated polymer material to the plasma environment within the chamber. The fluorinated polymer, positioned upstream of the frits being treated, decomposes under plasma conditions to produce fluorine-containing radicals that interact with the frit surfaces throughout the porous structure. This approach represents one preferred embodiment in the current experimental configuration.

More generally, the fluorine-containing plasma species may be generated from fluorinated gases, fluorinated polymers exposed to the plasma, or other fluorine-containing compounds introduced into the plasma environment. The invention is not limited to any particular source of fluorine-containing species, provided that the resulting plasma modification reaches the internal pore surfaces of the porous frit and produces a surface that improves adhesion and uniformity of subsequent coatings applied to the frit, reduces non-specific interaction between analytes and the underlying frit substrate, and remains stable for extended periods prior to coating.

Incorporating oxygen or other reactive gases optionally with the fluorine-containing plasma species during plasma treatment enhances surface modification and allows control over surface energy characteristics in accordance with various embodiments. Notably, oxygen plasma alone does not effectively modify the internal surfaces of the porous frit and the treatment tends to decay relatively quickly. When fluorine-containing species are present in the plasma environment, the modification penetrates much more effectively throughout the porous frit structure and remains stable for extended periods prior to coating. Oxygen or other reactive gas content is not limited to a narrow low-percentage range and may vary from trace or minor amounts to high oxygen content, including substantially oxygen-rich or effectively 100% oxygen feed conditions where fluorinated species are generated from a fluorinated source such as a PTFE target exposed to the plasma environment. The addition and relative amount of reactive gases may be adjusted to optimize the interface between the frit substrate and the subsequently applied stationary phase coating.

In a preferred embodiment of the invention, the plasma fluorination process parameters comprise operating conditions selected to achieve reproducible surface modification without unnecessarily limiting the process to a single narrow parameter window. The plasma treatment may be conducted in a capacitively-coupled discharge system, inductively-coupled plasma system, microwave plasma system, atmospheric plasma system, or other plasma-generation system at pressures, power levels, gas compositions, and exposure times selected based on the frit material, frit geometry, plasma source, fluorine-containing species source, target coating chemistry, desired surface energy, and desired degree of surface modification. In some embodiments, chamber pressure may be between about 0.01 Torr and about 10 Torr, RF or other plasma power may be between about 10 watts and about 1000 watts, and exposure time may range from a few seconds to several hours. Longer exposures may be used where deeper or more durable surface modification, increased fluorinated functionality, increased surface roughening, or a particular coating-adhesion profile is desired, provided that process conditions are selected to avoid unacceptable degradation, melting, embrittlement, or pore blockage of the base polymer.

The chamber conditions in another embodiment are controlled to maintain plasma stability, with gas flow rates, target position, target surface area, target condition, plasma power, pressure, exposure time, and substrate placement selected to produce the desired fluorinated surface modification. Fluorine-containing reactive species may be generated from fluorine-containing gas flow, from a fluorinated polymer or fluorinated target such as PTFE exposed to the plasma, from another fluorine-containing material in the chamber, or from combinations thereof. Where oxygen or other reactive gases are included, their proportion may be varied broadly, including oxygen-rich or effectively 100% oxygen feed conditions in embodiments in which fluorinated species are supplied by a PTFE target or other fluorinated source rather than solely by a fluorinated gas. The actual reactive species composition in the plasma need not be defined by a fixed percentage because it depends on variables including PTFE target surface area, PTFE target age or surface condition, oxygen flow rate, fluorinated gas flow rate if present, plasma power, chamber pressure, exposure time, electrode configuration, and the location of the frits relative to the plasma and fluorinated source. The chamber temperature is preferably controlled to prevent thermal damage to the polymer frit, for example by limiting substrate heating, pulsing the plasma, cooling the chamber, or otherwise controlling process heat load.

In a preferred embodiment, the plasma treatment parameters are verified through surface wettability measurements. Because plasma-fluorinated surfaces are highly hydrophobic and chemically distinct from surfaces treated with oxygen-based plasmas, a test solution chemically analogous to the sorptive polymer formulation is used to evaluate wetting behavior. The process parameters are adjusted to achieve optimal surface compatibility for subsequent coating adhesion, as indicated by consistent wetting of the polymer solution across the frit surface. Contact angle measurements using the sorptive polymer solution should demonstrate angles less than 45 degrees, confirming effective surface modification and wetting properties. Modifying surface energy through plasma fluorination produces several key effects. The process introduces fluorinated chemical groups, such as carbon-fluorine (C—F) and fluorocarbon (CFₓ) functionalities, onto the surface of the sintered polymer frit. These modifications reduce surface energy and enhance hydrophobicity while also improving the adhesion of subsequently applied polymer coatings. In certain plasma conditions, localized surface etching and mild crosslinking may also occur, leading to increased surface roughness (rugosity) that further promotes mechanical interlocking of coatings. This combination of chemical and topographical changes enables consistent, uniform polymer deposition while minimizing non-specific analyte interactions with the base material.

In an exemplary embodiment, the surface energy modification serves dual purposes by reducing non-specific analyte binding to the base frit substrate while enhancing polymer coating adhesion. The modified surface enables reproducible, conformal coating throughout the internal frit geometry.

Plasma fluorination of the frit surface optimizes wetting conditions for the subsequent sorptive polymer coating. Although plasma fluorination generally lowers absolute surface energy compared to oxygen-based plasma treatments, it introduces fluorinated chemical groups that are chemically compatible with low-surface-energy polymers, such as polydimethylsiloxane. This chemical affinity promotes uniform wetting and spreading of the sorptive coating across the frit structure. Wetting performance can be quantitatively assessed using contact angle measurements between the sorptive polymer solution and the plasma-treated frit surface, where improved wetting is indicated by a lower contact angle. Plasma-fluorinated surfaces are highly hydrophobic and chemically distinct from standard high-energy surfaces. A wetting solution chemically analogous to the sorptive polymer formulation is used to verify proper surface modification and predict coating adhesion in accordance with various embodiments of the invention. The plasma fluorination process produces a surface that maintains complete physical homology while exhibiting enhanced coating adhesion properties. This allows the subsequent polymer coating to function as an immobilized liquid that remains completely immiscible in aqueous sample matrices, ensuring clean back-extraction when simple preventative measures are taken.

In a preferred embodiment of the invention, the sorptive polymer coating comprises specific characteristics that enable effective analyte extraction. The coating is composed of an elastomeric polymer that remains above its glass transition temperature at room temperature, allowing it to exhibit the extractive characteristics of a liquid while maintaining complete physical homology.

In an embodiment, the sorptive polymer is selected from the group consisting of polydimethylsiloxane (PDMS), polybutadiene, siloxane copolymers, polydivinylbenzene (PDVB), polyurethane elastomers, polyacrylates, polymethacrylates, polyacrylonitrile-based copolymers, cyano-functionalized polymers, flexible phenol-functionalized copolymers, polymeric ionic liquids (PILs), polycaprolactone (PCL), fluorinated polymer copolymers, polyethylene-co-polypropylene copolymers, polystyrene-butadiene copolymers (SBS), and ethylene-propylene-diene monomer (EPDM) rubbers. These polymers are selected for their ability to remain above their glass transition temperature at room temperature, allowing efficient analyte partitioning while maintaining phase stability. They are substantially immiscible with aqueous media such as serum, plasma, and urine, enabling consistent, reproducible extraction performance in biological matrices. Other alternative polymers bearing similar characteristics may be utilized in embodiments of the invention.

In varying embodiments of the invention, an alternative polymer that remains above its glass transition temperature at room temperature and exhibits the extractive characteristics of a liquid while maintaining complete physical homology may be used as the sorptive coating. Such polymers function as immobilized liquids that allow small molecules to partition into the extracting phase while remaining completely immiscible in aqueous sample matrices. This enables clean back-extraction when appropriate preventative measures are taken. The polymer's ability to maintain complete physical homology while exhibiting liquid-like diffusion characteristics is critical for achieving consistent extraction performance across diverse analytical applications.

The coating thickness in a preferred embodiment is approximately 3 microns, applied uniformly across the internal surface area of the frit. This specific thickness is optimized to provide sufficient sorptive capacity while maintaining rapid diffusion kinetics. If the coating is too thick, the surface becomes loaded and analytes must move through the polymer before more can be added, which slows down the extraction process.

In another embodiment, the polymer coating exhibits specific glass transition temperature requirements, remaining above its glass transition temperature at room temperature to enable efficient analyte uptake. The most usable immobilized liquid polymers are those that exhibit an open morphology and have a backbone that easily rotates, typically requiring a glass transition temperature below 0° centigrade with minimal crosslink density.

The surface coverage parameters are critical for performance. In a preferred embodiment, the coating provides complete coverage of the internal frit surfaces while maintaining the porous structure. Small molecules are soluble in the extracting phase, allowing partitioning to occur when an aqueous matrix is directly exposed to the polymer. The non-porous nature of the immobilized liquid coating eliminates problems often encountered with existing methods such as the formation of emulsions, analyte breakthrough, and clogging.

In a preferred embodiment of the invention, the coating application process comprises applying the sorptive polymer using a mixed solvent/anti-solvent system through the following method and associated steps:

Preparing the coating solution by dissolving the sorptive polymer in a solvent to form a primary solution, then adding an anti-solvent in an amount sufficient to reduce the solubility of the polymer while maintaining a metastable single-phase solution.

Applying the coating solution to the plasma-fluorinated frit, allowing the polymer to precipitate during solvent and anti-solvent evaporation as a conformal coating. The evaporation is performed at ambient temperature or under mild heat below 50 °C to control the rate of polymer deposition.

Selecting solvents and anti-solvents wherein the solvent is chosen from hydrocarbons, ethers, esters, or alcohols, and the anti-solvent is selected from water, alkanes, or short-chain alcohols. The anti-solvent is specifically selected to lower the solubility of the sorptive polymer without inducing immediate precipitation prior to evaporation.

In an embodiment, the plasma fluorination alters surface energy to facilitate uniform coating via the solvent/anti-solvent system. This surface modification enables reproducible, conformal internal coating throughout the frit geometry.

The coating process produces a thin layer approximately 3 microns thick that covers the entirety of the internal surface area of the frit. The process is optimized to achieve complete coverage while maintaining the porous structure and flow characteristics of the frit.

In another embodiment, the coating process includes stabilization or curing steps to ensure proper adhesion and coating integrity. The process parameters are controlled to achieve batch consistency and coating uniformity across production runs. Quality control testing verifies proper coating thickness, uniformity, and adhesion to the plasma-treated frit surface.

In a preferred embodiment of the invention, the solvent/anti-solvent system comprises specific solvents selected to achieve optimal polymer deposition and coating uniformity. The solvent is selected from the group consisting of hydrocarbons, ethers, esters, or alcohols, while the anti-solvent is selected from water, alkanes, or short-chain alcohols.

The process involves dissolving the sorptive polymer in a solvent to form a primary solution, then adding an anti-solvent in an amount sufficient to reduce the solubility of the polymer while maintaining a metastable single-phase solution. The anti-solvent is specifically selected to lower the solubility of the sorptive polymer without inducing immediate precipitation prior to evaporation.

In an embodiment, the polymer precipitates during solvent and anti-solvent evaporation as a conformal coating. The evaporation is performed at ambient temperature or under mild heat below 50 °C to control the rate of polymer deposition, allowing the sorptive polymer to precipitate as a uniform coating on internal and external surfaces of the frit.

The plasma fluorination treatment of the frit surface enhances the effectiveness of the solvent/anti-solvent system by altering surface energy to facilitate uniform coating. This surface modification enables reproducible, conformal internal coating throughout the frit geometry.

In a preferred embodiment of the invention, the solvent/anti-solvent coating system comprises specific ratios and parameters for achieving consistent coating properties. The primary solution is prepared with a polymer concentration of 0.5-5.0% w/v in the solvent phase. The anti-solvent is added in a ratio of 1:2 to 1:4 relative to the primary solution while maintaining a metastable single-phase system.

For elastomeric polymers like AEM copolymers, the primary solvent comprises a mixture of hydrocarbons, ethers, or esters, with the anti-solvent selected from water or short-chain alcohols. The critical polymer concentration range is maintained between 1-3% w/v to achieve proper coating viscosity and thickness control.

The process parameters for achieving the specified 3-micron coating thickness include carefully controlled deposition conditions. The coating solution is applied at room temperature (20-25 °C) with controlled relative humidity below 60%. The evaporation rate is regulated through mild heating at 30-45 °C while maintaining laminar airflow conditions.

Quality control measurements verify coating thickness using microscopic examination of cross-sections and gravimetric analysis. The coating weight per unit surface area is maintained within ±10% of the target specification across the internal frit geometry.

In a preferred embodiment, the solvent/anti-solvent ratios and evaporation conditions are controlled to achieve consistent coating thickness of approximately 3 microns across the internal surface area of the frit. The system enables complete coverage while maintaining the porous structure and flow characteristics of the frit.

In a preferred embodiment of the invention, the solution preparation methods comprise specific steps for creating the coating solution. The process begins by dissolving the sorptive polymer in a primary solvent selected from hydrocarbons, ethers, esters, or alcohols to achieve complete dissolution.

In an embodiment, the solution preparation includes careful control of polymer concentration and solvent ratios to achieve optimal coating properties. The anti-solvent is added in precise amounts to reduce polymer solubility while maintaining a metastable single-phase solution. This balance is critical as the anti-solvent must lower the solubility of the sorptive polymer without causing immediate precipitation.

The solution preparation process requires controlling temperature and mixing conditions to ensure uniform polymer distribution. In a preferred embodiment, the polymer solution is prepared at ambient temperature or under mild heating conditions below 50 °C to maintain solution stability while enabling proper coating deposition.

For commercial pre-polymers used in solution preparation, the process includes polymer fractionation and cleaning steps. The pre-polymers typically have a large molecular weight dispersion range that must be controlled. Smaller molecular weight fractions that lack sufficient crosslinking functionality are eliminated to prevent them from becoming extractable components in the final product.

In a preferred embodiment of the invention, the polymer cleaning and fractionation process comprises specific methods for removing low molecular weight components and processing aids from commercial pre-polymers. The fractionation process begins by dissolving the pre-polymer in a suitable solvent at 1-5% concentration by weight. The solution is then subjected to selective precipitation using an anti-solvent to remove low molecular weight fractions that lack sufficient crosslinking functionality.

In an embodiment, the fractionation is conducted through controlled addition of the anti-solvent while maintaining the temperature between 20-30 °C. The precipitated high molecular weight fraction is collected by filtration or centrifugation, while the low molecular weight components remain in solution. This process may be repeated multiple times to achieve the desired molecular weight distribution.

The removal of processing aids in a preferred embodiment is accomplished through washing steps using appropriate solvents. For AEM copolymers, the polymer is washed sequentially with methanol and acetone to remove processing aids while maintaining the polymer's physical properties. The washing process continues until spectroscopic analysis confirms removal of processing aid components.

In another embodiment, the cleaned and fractionated polymer is dried under vacuum at temperatures below 50 °C until constant weight is achieved. The final product is analyzed for molecular weight distribution using gel permeation chromatography to verify removal of low molecular weight fractions. The cleaned polymer should contain less than 1% of components below the critical molecular weight for entanglement.

In another embodiment, the solution preparation includes removal of processing aids and other contaminants from the pre-polymers prior to coating solution formulation. This cleaning step is essential as these components are considered contamination in the final product and must be eliminated to ensure coating quality and performance.

In a preferred embodiment of the invention, the application techniques for the sorptive polymer coating comprise specific methods for achieving uniform coverage of the frit surface. The coating is applied to the plasma-treated frit using controlled dispensing of the polymer solution, allowing even distribution throughout the porous structure.

In an embodiment, the application process involves carefully controlling the coating environment temperature and conditions. The evaporation is performed at ambient temperature or under mild heat below 50 °C to control the rate of polymer deposition and achieve uniform coating formation.

The application technique in a preferred embodiment utilizes the modified surface energy from plasma fluorination to facilitate uniform coating via the solvent/anti-solvent system. This surface modification enables reproducible, conformal internal coating throughout the frit geometry while maintaining the porous structure.

In another embodiment, the coating is applied by allowing the polymer to precipitate during controlled evaporation of the solvent and anti-solvent, forming a uniform coating on both internal and external surfaces of the frit. The precipitation process is carefully managed to achieve a coating thickness of approximately 3 microns across the entirety of the internal surface area.

The application technique includes verification steps to ensure proper coating coverage and uniformity. In a preferred embodiment, the coating process is monitored to confirm complete coverage of the internal frit surfaces while maintaining the porous structure and flow characteristics. The non-porous nature of the applied coating eliminates problems often encountered with existing methods such as emulsion formation, analyte breakthrough, and clogging.

In a preferred embodiment of the invention, the evaporation and curing conditions are carefully controlled to achieve optimal coating properties. The evaporation is performed at ambient temperature or under mild heat below 50 °C to control the rate of polymer deposition, allowing the sorptive polymer to precipitate as a uniform coating on internal and external surfaces of the frit.

In an embodiment, the evaporation conditions are managed to maintain a metastable single-phase solution until the coating process is complete. The anti-solvent is selected to lower the solubility of the sorptive polymer without inducing immediate precipitation prior to evaporation, enabling controlled deposition of the coating.

For elastomeric polymers like AEM copolymers, the curing conditions can be controlled through either peroxide free radical systems or primary diamine vulcanization. In a preferred embodiment using AEM terpolymers, the vulcanization requires an extensive cure and post cure to complete the reaction and convert amide cross links to imide crosslinks.

In a preferred embodiment of the invention, the vulcanization and curing processes for elastomeric polymers comprise specific conditions and parameters for achieving optimal crosslinking. For AEM copolymers, the vulcanization can be controlled through either peroxide free radical systems or primary diamine vulcanization.

In an embodiment using AEM terpolymers, the vulcanization process requires specific temperature and time parameters. The initial cure is conducted at temperatures not exceeding 110 °C to remain below the melting point of the high-density polyethylene frit material (approximately 130 °C). The curing process is conducted for approximately 4 hours to achieve proper crosslinking.

The vulcanization and curing conditions in accordance with an embodiment must be carefully controlled to prevent degradation of the frit substrate while ensuring proper polymer crosslinking. The temperature parameters are specifically selected to maintain the structural integrity of the polyethylene frit while achieving adequate polymer curing.

For AEM copolymers in a preferred embodiment, the free radical crosslinking system targets the most labile hydrogen on the acrylate group, though some chain formation also occurs across the ethylene segments. The process requires careful control of peroxide concentration and cure temperature to achieve optimal crosslink density while maintaining the polymer's physical properties.

The shelf stability of the cured polymers in a preferred embodiment is excellent, being measured in years. The cured polymers maintain their rubber-like qualities for extended periods, with useful life varying based on exposure temperature. At 121 °C, the approximate useful life is two years, decreasing to 5 days at 204 °C

In another embodiment, the evaporation and curing conditions are optimized to achieve a coating thickness of approximately 3 microns while maintaining complete coverage of the internal frit surfaces. The process parameters ensure the non-porous nature of the immobilized liquid coating, which eliminates problems often encountered with existing methods such as emulsion formation and analyte breakthrough.

In a preferred embodiment of the invention, the pipette tip implementation comprises specific requirements for integrating the coated frit into standard pipette tips. The frit is inserted into the conus of a 1 mL pipette tip and pressed into the area just prior to the tip transition point.

The insertion specifications in an embodiment require precise positioning of the cylindrical porous frit with dimensions of 4.5 mm×6.5 mm. The frit is inserted into a pipette tip, creating an extraction device that maintains proper flow characteristics while enabling effective sample preparation.

In a preferred embodiment, the positioning requirements ensure the frit is securely placed at the tip transition point. This specific positioning is critical for maintaining proper flow paths and enabling consistent extraction performance. The frit must be pressed firmly into position to prevent any bypass flow around the edges.

The flow path considerations in an embodiment focus on optimizing the three-dimensional geometry to enable both convective and diffusive transport of analytes into the sorptive coating under dynamic and static conditions. The positioning and dimensions of the frit are designed to maintain low dead volume while providing sufficient surface area for extraction.

In another embodiment, the flow path design enables a simplified three-step workflow: extraction, rinse, and elution. The extraction is accomplished by aspirating and dispensing a sample three times over 50 seconds, followed by rinsing with a wash solution for 20 seconds, and eluting with solvent using three aspiration/dispense cycles over 50 seconds. The oscillatory aspirate/dispense operation provides a significant mass-transfer advantage when used with the porous frit architecture. Because the stationary phase resides on the internal pore surfaces of the frit, each aspirate/dispense cycle causes liquid to move through the interconnected pore network, promoting mixing within the pores and continually refreshing the liquid in contact with the stationary phase surfaces. As a result, analytes interact with the stationary phase multiple times during a single processing step. This behavior differs from conventional packed SPE cartridges or chromatography columns, which typically rely on unidirectional flow through a particle bed. In the present architecture, oscillatory aspirate/dispense motion enhances interaction kinetics and improves extraction efficiency while using relatively small sample volumes, enabling efficient analyte interaction during relatively short processing cycles.

The implementation in a preferred embodiment allows for integration with both manual and automated liquid handling systems. The device maintains consistent performance across multiple aspiration and dispense cycles while preventing issues like clogging or emulsion formation that are common with traditional extraction methods.

In a preferred embodiment of the invention, the frit insertion process comprises specific methods and parameters for achieving proper positioning and sealing. The cylindrical porous frit with dimensions of 4.5 mm×6.5 mm is inserted into the conus of a 1 mL pipette tip using controlled force to achieve precise positioning at the tip transition point.

The positioning requirements in a preferred embodiment specify that the frit must be pressed firmly into the area just prior to the tip transition point. The insertion depth is controlled to ensure the frit creates a compression seal against the internal walls of the pipette tip conus while maintaining proper flow path geometry. This specific positioning prevents any bypass flow around the edges while enabling both convective and diffusive transport of analytes.

In another embodiment, the sealing parameters are verified through flow resistance testing. The properly sealed frit demonstrates consistent flow resistance within ±15% of specification when tested with standard solutions. The compression fit creates a fluid-tight seal between the frit's outer diameter and the pipette tip's inner wall, preventing any sample from bypassing the sorptive coating.

Quality control measures in a preferred embodiment include visual inspection of frit positioning using calibrated measurement tools to verify proper insertion depth. Flow path testing confirms uniform sample distribution across the frit cross-section without channeling or bypass. Each assembled device undergoes leak testing using pressurized air or liquid to verify seal integrity.

The assembly process in another embodiment includes verification of proper frit orientation to maintain optimal flow characteristics. The frit's cylindrical geometry must be properly aligned with the pipette tip axis to ensure uniform sample contact with the sorptive coating. Quality control testing confirms consistent extraction performance across multiple aspiration and dispense cycles, demonstrating proper frit positioning and sealing.

In a preferred embodiment of the invention, the coated frit can be integrated into various device implementations beyond pipette tips. For cartridge systems, the frit may be incorporated into standard SPE cartridges along with flow through well plates. The cartridge implementations include luer tip or luer lock fittings for secure connections.

In an embodiment utilizing spin columns, the sorptive frit is placed at the bottom exit of the device. The spin column configuration enables centrifugation-based sample processing while maintaining the extraction capabilities of the coated frit.

For microfluidic device implementations, the invention can be integrated into lab-on-chip or other microfluidic systems. The three-dimensional geometry and flow characteristics of the coated frit enable effective integration into microfluidic channels while maintaining extraction performance.

In a preferred embodiment, the flow-through configurations include filter funnels and other continuous flow devices. These implementations maintain the porous and flow-through operation capabilities of the frit while enabling convective mass transport during analyte extraction.

The device implementations in another embodiment include cartridge, syringe, or analytical flow cell configurations. The frit is manufactured as a modular plug-in compatible with standard fluidic connections, enabling integration into disposable or automated sampling systems.

For filter well plate implementations, various filtration components may be incorporated into the device according to different embodiments of the invention. In one exemplary embodiment, prefilter and membrane components are replaced by the sorptive filter frit. More broadly, the invention contemplates the use of any suitable filtration element or combination of elements that can be integrated into filter well plates, including but not limited to frits, membranes, filters, or combinations thereof. Regardless of the specific filtration components employed, the configuration maintains proper flow characteristics while enabling effective sample preparation through the three-step extraction, rinse, and elution process.

In a preferred embodiment of the invention, the sample preparation parameters are optimized for effective analyte extraction. The sample preparation may involve combining an extraction mix with a biological sample in proportions that facilitate optimal extraction efficiency. In an exemplary embodiment, the total sample volume is approximately 200 μl, which may comprise approximately 150 μl of extraction mix combined with approximately 50 μl of serum or other biological sample. However, the invention contemplates a range of sample volumes and ratios that may be optimized based on the specific analyte, matrix, and analytical requirements of a particular application.

In another embodiment, the sample preparation includes combining a modifier buffer with a sample in a ratio that optimizes extraction conditions. The ratio between modifier buffer and sample may vary depending on analyte properties, matrix complexity, and desired analytical performance. As an example, one implementation may utilize approximately 100 μl of modifier buffer combined with approximately 100 μl of sample. The modifier buffer typically contains internal standards and pH modifiers to optimize extraction conditions, with the specific composition tailored to the requirements of the target analytes and sample matrices.

The invention demonstrates compatibility with a broad range of biological fluid matrices including blood, plasma, serum, urine, saliva, cerebrospinal fluid, sputum, synovial fluid, amniotic fluid, breast milk, sweat, tears, and cell lysates prepared from cultured cells or tissue homogenates. The extracting phase of the sorptive polymer coating and the functional chemistries applied to the discrete porous processing modules are substantially immiscible with or chemically distinct from these aqueous biological matrices, enabling clean analyte capture and elution across the full range of matrix types. The device architecture is particularly well suited to low-volume biological fluids such as cerebrospinal fluid and synovial fluid, where sample volume constraints are significant, because the low dead volume of the porous frit modules and the short diffusion paths within the interconnected pore network enable efficient analyte extraction from sample volumes as small as 50 microliters. Saliva represents an additional clinically relevant matrix for therapeutic drug monitoring, toxicology screening, and biomarker discovery applications, and the device has been shown to be compatible with the viscosity and protein content typical of whole saliva samples. The extracting elastomer is completely immiscible in these aqueous sample matrices, which ensures clean back-extraction when appropriate rinsing steps are performed.

The invention demonstrates matrix compatibility through successful extraction of testosterone from human serum with a lower limit of quantitation of 0.04 ng/ml, THC metabolites from urine with excellent reproducibility achieving a coefficient of variation of 5.6%, and Vitamin D metabolites from serum with high accuracy of ±10% and precision with coefficient of variation less than 9%.

The analyte requirements in a preferred embodiment specify compounds with LogP greater than 1, indicating sufficient hydrophobicity for effective extraction. The extraction process is particularly effective for small organic molecules from complex aqueous matrices. The partition ratio of an analyte for the extraction phase compared to water can be predicted based on the compound's LogP value, allowing for estimation of extraction efficiency.

The invention demonstrates successful extraction across a range of LogP values, including testosterone with LogP 3.365, THC-COOH with LogP 5.14, and 25-OH-Vitamin D2 with LogP 7.051, showing broad applicability across different analyte types.

In a preferred embodiment of the invention, the operating procedures comprise specific extraction, rinse and elution steps. The extraction step involves aspirating and dispensing the sample mixture three times over 50 seconds. This extraction timing allows analytes to partition into the sorptive coating to reach equilibrium.

In another embodiment, the extraction process involves aspirating and dispensing a sample three times over a period of 20 seconds to achieve optimal extraction efficiency. The timing and number of cycles are critical for allowing sufficient contact between the sample and the sorptive coating while maintaining practical throughput.

The rinse protocol in a preferred embodiment comprises aspirating and dispensing a wash solution once over 20 seconds. The wash solution typically contains appropriate modifiers to remove residual matrix components while maintaining analyte retention. For example, when extracting testosterone from serum, the wash solution contains 0.2% formic acid in water.

In an embodiment for vitamin D metabolite extraction, the rinse step uses water/methanol (90:10 v/v) as the wash solution. This composition enables effective removal of matrix interference components without significant analyte loss.

The elution methods in a preferred embodiment involve aspirating and dispensing an elution solvent three times over 50 seconds. The elution solvent composition is optimized based on analyte properties. For THC metabolites, the elution uses water/acetonitrile (15/85 v/v) with 0.2% formic acid.

In another embodiment demonstrating the elution process, the eluted sample is transferred directly to an autosampler vial or plate fitted with a pierceable cover to minimize solvent evaporation. The eluate is then placed in a cooled autosampler (10 °C) for immediate analysis by LC-MSMS.

In a preferred embodiment of the invention, the performance characteristics demonstrate exceptional extraction efficiency and analytical performance. The extraction efficiency for testosterone and 13C3-testosterone using the IFX Extraction Tip method achieves 53 and 55% recovery respectively.

The IFX Extraction Tip is a pipette tip containing a porous frit that has been plasma fluorinated and coated with a sorptive polymer for extracting small molecules from biological samples. The device consists of a frit with dimensions of approximately 4.5 mm×6.5 mm that is inserted into the conus of a 1 mL pipette tip and pressed into the area just prior to the tip transition point. The frit is internally coated with a sorptive phase that functions like an immobilized liquid, allowing analytes to partition into the polymer coating rather than simply adsorbing to the surface. The extraction process using the IFX Extraction Tip follows a simple three-step workflow: aspirating and dispensing the sample three times over 50 seconds for extraction, performing a single rinse step over 20 seconds, and eluting with solvent through three aspiration/dispense cycles over 50 seconds. This device enables efficient sample preparation while eliminating common problems like clogging, emulsion formation, and analyte breakthrough that are typically encountered with traditional extraction methods.

In an embodiment demonstrating reproducibility, the extraction and analysis of testosterone from serum calibrators shows excellent consistency with coefficient of variation ranging from 1.0 to 8.3% for peak areas. The internal standard areas demonstrate high reproducibility with CV values between 1.0 and 7.5%.

Matrix effects in a preferred embodiment are effectively minimized, with testosterone extraction from serum samples showing only negligible ionization enhancement of 103%. This demonstrates the effectiveness of the rinse step in removing matrix interference components.

The analytical parameters in another embodiment show exceptional sensitivity and precision. For testosterone analysis, the lower limit of quantitation (LLOQ) is established at 0.04 ng/ml, with signal-to-noise ratio greater than 10. For THC-COOH analysis, the LLOQ is achieved at 2.5 ng/ml with excellent reproducibility.

In a preferred embodiment, the linear range demonstrates outstanding performance across multiple applications. Testosterone analysis shows linearity from 0.04 to 10 ng/ml with correlation coefficient (r2) greater than 0.998. THC-COOH analysis demonstrates linearity between 2.5 and 100 ng/ml with correlation coefficient (r) greater than 0.998.

The precision specifications in another embodiment show high reproducibility across different applications. For vitamin D metabolite analysis, high accuracy of ±10% and precision with CV less than 9% are achieved across calibration levels. Batch-to-batch variation analysis of antidepressants in plasma demonstrates average CV of approximately 5% across ten batches of 100 inserts each.

In a preferred embodiment of the invention, the manufacturing considerations include specific quality control parameters and production specifications. The batch consistency requirements demonstrate high reproducibility, with batch-to-batch variation analysis showing average coefficient of variation of approximately 5% across ten batches of 100 extraction devices.

In a preferred embodiment of the invention, the quality control testing methods comprise specific procedures for verifying coating properties and consistency. The coating uniformity testing includes microscopic examination of cross-sections and gravimetric analysis to verify the coating weight per unit surface area is maintained within ±10% of target specifications across the internal frit geometry. Surface coverage is confirmed through scanning electron microscopy to ensure complete coating of internal surfaces while maintaining the porous structure.

In another embodiment, batch consistency requirements specify acceptance criteria for manufacturing validation. Each production batch undergoes extraction efficiency testing using standard analytes, with acceptance requiring recovery rates within ±10% of established specifications. Reproducibility testing demonstrates coefficient of variation less than 10% for internal standard peak areas across multiple extractions. Batch-to-batch variation analysis requires average coefficient of variation of approximately 5% across ten batches of 100 extraction devices.

The quality control process in a preferred embodiment includes verification of coating thickness using multiple measurement techniques. Cross-sectional analysis confirms the 3-micron target thickness is maintained within ±0.5 microns. Flow resistance measurements verify consistent pore structure and coating uniformity, with acceptance criteria requiring less than 15% variation in flow rates across the batch. Each production batch undergoes matrix effect testing using standard biological samples, with acceptance requiring ionization enhancement or suppression within ±5% of established specifications.

The coating uniformity testing in an embodiment includes verification of complete coverage across internal frit surfaces while maintaining porous structure. The coating thickness is verified to be approximately 3 microns across the entirety of the internal surface area. Quality control testing confirms proper coating adhesion to the plasma-treated frit surface.

In a preferred embodiment, the frit dimensional tolerances specify a cylindrical porous frit with dimensions of 4.5 mm×6.5 mm. These dimensions are critical for proper insertion into the pipette tip conus and maintaining appropriate flow characteristics.

The coating process controls in another embodiment include careful management of the solvent/anti-solvent system. The evaporation is performed at ambient temperature or under mild heat below 50 °C to control polymer deposition rate. The anti-solvent concentration is controlled to reduce polymer solubility while maintaining a metastable single-phase solution.

Assembly requirements in a preferred embodiment specify insertion of the coated frit into the conus of a 1 mL pipette tip, with precise positioning at the tip transition point. The frit must be pressed firmly into position to prevent bypass flow while maintaining proper flow path characteristics. The assembly process ensures the device is compatible with both manual and automated liquid handling systems.

The present invention in various embodiments thus provides a revolutionary approach to sample preparation and analyte extraction through its innovative combination of plasma-fluorinated porous frits and sorptive polymer coatings. The invention's unique integration of surface treatment and coating technology enables reproducible, conformal coating throughout the frit geometry while minimizing non-specific binding.

The device demonstrates exceptional performance across multiple applications, achieving lower limits of quantitation of 0.04 ng/ml for testosterone, 2.5 ng/ml for THC-COOH, and consistent detection of vitamin D metabolites down to 4.2 ng/ml. This high sensitivity is complemented by excellent reproducibility, with coefficient of variation typically around 5% across multiple batches.

The invention's key advantages include immunity to clogging from complex biological samples, elimination of emulsion formation, and prevention of analyte breakthrough due to its equilibrium-based extraction mechanism. The device requires no preconditioning or pre-wetting steps, enables effective matrix rinsing for clean extracts, and significantly reduces solvent usage compared to traditional methods.

The modular design allows integration into various analytical platforms, from simple pipette tips to complex automated systems. The three-step workflow-extraction, rinse, and elution-can be completed in under two minutes, representing a significant improvement over traditional 20-30 minute protocols.

The invention's plasma fluorination treatment serves the dual purpose of reducing non-specific analyte binding while enhancing coating adhesion. This surface modification, combined with the sorptive polymer coating above its glass transition temperature, enables efficient extraction of small molecules while excluding larger interfering compounds.

The technology demonstrates broad applicability across different analyte types and matrices, successfully extracting compounds with LogP values ranging from 3.365 to 7.051. This versatility, combined with high reproducibility and simple automation potential, positions the invention as a significant advancement in sample preparation technology.

In an embodiment of the invention, the coating applied to the plasma-fluorinated frit may comprise surface interaction chemistries that operate through mechanisms distinct from the sorptive polymer approach described above. These surface interaction systems enable specific molecular recognition through targeted chemical interactions rather than bulk dissolution of analytes into a polymer phase.

An exemplary surface interaction chemistry comprises tunable surface interaction functionalities that provide selective binding sites for particular analyte classes. Unlike sorptive coatings where analytes dissolve into the bulk polymer, surface interaction chemistries provide discrete binding sites located on the frit surface where analytes interact through hydrogen bonding, π-π stacking, electrostatic interactions, hydrophobic interactions, dipole interactions, coordination interactions, affinity interactions, or other specific molecular recognition mechanisms. Interaction strength and selectivity may be tuned by selecting chain length, functional groups, ligand density, polymer composition, crosslink density, charge state, hydrophobicity, aromaticity, or combinations thereof, and different modules can be designed for different interaction types.

In a preferred implementation of an embodiment of the invention, the surface interaction chemistry is applied using solvent-based coating systems that differ from the solvent/anti-solvent precipitation approach. The solvent-based system dissolves the functional chemistry in an appropriate solvent and applies it directly to the plasma-treated frit surface, where it forms a thin, uniform layer through controlled evaporation or chemical cross-linking.

An embodiment of the invention further contemplates multi-modal coatings that combine both sorptive and surface interaction mechanisms within a single frit module. Such multi-modal systems provide enhanced selectivity by enabling both specific molecular interactions and bulk partitioning effects, thereby expanding the range of analytes that can be effectively extracted.

The manufacturing process according to various embodiments includes extensive quality control measures to ensure consistent coating coverage and performance. Plasma parameter optimization is conducted based on the specific frit material composition and the intended coating type, with different parameters required for surface interaction chemistries versus sorptive polymer systems.

Quality control methods for verifying coating uniformity include microscopic examination of frit cross-sections, scanning electron microscopy of internal surfaces, and gravimetric analysis to confirm coating mass per unit surface area. Surface contact angle measurements using test solutions chemically analogous to the intended application verify proper surface modification and coating adhesion.

Scale-up considerations for commercial manufacturing include batch consistency monitoring, automated plasma treatment systems with real-time process monitoring, and statistical process control methods to maintain coating specifications across production runs. Alternative plasma gases beyond those initially described may include nitrogen trifluoride (NF₃), difluoromethane (CH₂F₂), and trifluoromethane (CHF₃), each providing different surface modification characteristics.

According to preferred embodiments of an embodiment of the invention, modular stacked configurations comprise multiple frit modules positioned sequentially within a single device housing. Each frit module contains distinct functional chemistry tailored for specific analytical operations, enabling complex multi-step sample preparation workflows within a single disposable device.

The stacked modules are dimensioned to seal against the interior walls of the housing, preventing bypass flow around individual modules. This sealing arrangement ensures that all sample fluid must pass through each frit module sequentially, enabling precise control over the analytical workflow.

Void chambers may be incorporated between frit modules to provide isolation and staging between adjacent functional chemistries. These void chambers separate frits, prevent liquid from one frit from immediately contacting the next frit, and allow the timing of contact with downstream chemistry to be controlled. In preferred aspirate/dispense workflows, liquid may be dispensed to an external well or vessel for pH adjustment, solvent adjustment, reagent addition, incubation, or other conditioning before being aspirated back into the device. In ported or otherwise interfaced embodiments, reagent introduction or condition adjustment may occur directly in the void chamber.

An exemplary three-module configuration includes: a first module containing affinity ligands for analyte capture, a second module containing immobilized enzymes for biochemical transformations, and a third module containing reversed-phase chemistry for purification of reaction products. This configuration enables capture-react-purify workflows to be completed within a single disposable device.

An embodiment of the invention contemplates integration into various analytical platforms beyond simple pipette tips. Microfluidic integration specifications include dimensional tolerances compatible with standard microfluidic channel geometries and flow rate specifications appropriate for lab-on-chip applications.

Automated liquid handling compatibility requires standardized fluidic connections such as luer-lok fittings, threaded connections, or press-fit interfaces that maintain leak-tight seals under typical operating pressures. The devices may be supplied as individual modules or pre-assembled cartridges compatible with robotic liquid handling systems.

In embodiments of the invention employing a multi-well format with a plurality of staged analytical processing devices, individual devices within the same multi-well plate or rack may be independently configured with different combinations of discrete porous processing modules, thereby enabling parallel processing of different analytical protocols across the multi-well format within a single automated run. For example, a first subset of devices within the multi-well format may be configured with a first module combination providing a proteomics workflow comprising ion exchange capture, enzymatic digestion, and peptide cleanup, while a second subset of devices within the same multi-well format is configured with a second module combination providing a small molecule extraction workflow comprising reversed-phase capture and cleanup. A third subset may be configured with an affinity capture module followed by a purification module for targeted protein biomarker extraction. The automated liquid handling apparatus is programmed to apply workflow-appropriate aspiration and dispensing parameters, including flow rate, cycle number, and dwell time, independently to each subset of devices based on the module configuration of that subset. This capability allows a single automated run to generate processed samples suitable for multiple different analytical methods from different sample types or using different extraction chemistries without manual reconfiguration of the liquid handling system between runs. The independence of module configuration across devices within the same multi-well format is a direct consequence of the modular single-device architecture, wherein each device is a self-contained staged processing unit whose functional behavior is determined entirely by the module combinations installed within its housing.

Cartridge system implementations include housing materials compatible with typical laboratory solvents, proper dimensional tolerances for automated handling equipment, and standardized labeling or coding systems for automated identification of different module types.

Spin column operational parameters include balancing requirements for centrifugal processing, proper fit within standard centrifuge rotors, and mechanical integrity under typical centrifugal forces ranging from 1,000 to 16,000×g.

Conventional solid phase extraction devices described in the prior art, including those disclosed in U.S. Pat. Nos. 6,566,145, 8,715,593, and 2008/0119637, fundamentally rely on particulate sorbent materials retained by frits, where the frits function merely as mechanical barriers or filters to contain loose resin beads, silica particles, or polymeric extraction media. In these particle-retained-by-frit architectures, analytes must diffuse through potentially long pathways within porous particles to reach active sites, creating diffusion limitations that slow extraction kinetics and may result in incomplete analyte recovery, particularly for larger molecules or complex samples. For example, some systems disclose sorbent material contained loosely enough to move within a mixing chamber. Movement of loose particles may improve mixing, but the extraction mechanism remains dependent on commercial or conventional sorbent particles retained by barriers. The present device instead uses a porous module as an active flow-through stationary phase. The module is dimensioned to seal against the housing and to force sample through functionalized interconnected pores, thereby combining containment, flow distribution, and analytical chemistry in a single structure rather than allocating those functions to separate particles and passive retainers.

In conventional SPE materials such as silica-based C18 phases, the stationary phase is typically prepared by derivatizing mesoporous silica particles with silane reagents to produce bonded ligands. In these materials the stationary phase resides within the mesoporous structure of the silica particles, and the packed sorbent bed must usually be conditioned prior to use in order to properly wet the particles and ensure that the bonded ligands are accessible to the mobile phase. In contrast, the frit architecture of the present invention presents the stationary phase on internal pore surfaces that are directly accessible within the interconnected pore network of the porous frit. Because the liquid phase flows directly through these pores, the stationary phase can be readily contacted by the sample solution without requiring the conventional conditioning steps typically associated with packed silica SPE materials. As a result, certain embodiments of the frit-based system can operate without the preconditioning steps normally required for conventional SPE cartridges. This difference arises from the geometry of the porous frit and the accessibility of the stationary phase on the internal pore surfaces rather than from any specific stationary phase chemistry.

In contrast, an embodiment of the invention utilizes the porous frit itself as the structural chromatographic medium, wherein the stationary phase resides directly on the internal pore surfaces of the frit rather than within or upon retained particles. This fundamental architectural departure eliminates the need for particulate sorbents entirely, instead providing functional chemistry directly on the internal pore surfaces of the frit where analytes can interact without the long diffusion paths characteristic of particle-based systems. Because the interconnected pore structure of the sintered thermoplastic frit provides very short diffusion paths between the mobile phase and the stationary phase, the system achieves rapid interaction kinetics compared with conventional packed stationary phases. The frit-as-stationary-phase configuration provides shorter diffusion paths between analytes and functional sites compared to conventional packed-bed extraction media, enabling rapid interaction kinetics and improved analytical performance.

The stationary phase on the internal pore surfaces of the frit may be introduced through several approaches depending on the chemistry being implemented. These approaches include, without limitation, polymer deposition from solution, covalent surface modification using silane or other coupling reagents, sol-gel formation on the pore surfaces, graft polymerization initiated from the pore surfaces, immobilization of enzymes or other biomolecules, adsorption or covalent attachment of affinity ligands, and coordination of metal ions for chelation-based separations. In all cases the stationary phase resides on the internal pore surfaces of the frit, which allows analytes to interact with the phase throughout the pore network while maintaining very short diffusion distances between the mobile phase flowing through the pores and the stationary phase on the pore surfaces.

The functional chemistry borne by a porous processing module may be selected to perform any analytical separation or processing operation compatible with the sample and downstream analytical method. Non-limiting examples include C4, C8, C18, phenyl, pentafluorophenyl, cyano, diol, normal phase, reversed phase, hydrophilic interaction, strong cation exchange, weak cation exchange, strong anion exchange, weak anion exchange, mixed-mode ion exchange/reversed phase, boronate affinity, immunoaffinity, aptamer affinity, immobilized metal affinity, size exclusion, molecular-weight cutoff, hydrophobic interaction, enzymatic digestion, chemical derivatization, reduction, alkylation, desalting, buffer exchange, matrix depletion, phosphopeptide enrichment, glycopeptide enrichment, and polymeric or elastomeric sorptive partitioning chemistries.

Different modules within the same device may bear different ones of these chemistries so that the device performs a defined sequence of operations. For example, a first module may remove proteins or lipids from a biological matrix, a second module may capture an analyte or analyte precursor, a functional staging region may adjust pH or solvent strength, and a downstream module may elute, digest, desalt, derivatize, or polish the processed sample before collection or instrumental analysis.

In an embodiment of the invention, at least one discrete porous processing module bears a functional chemistry comprising an immobilized metal affinity chemistry applied to the internal pore surfaces of the porous body. Immobilized metal affinity chromatography, commonly referred to as IMAC, involves the coordination of metal ions such as nickel, cobalt, copper, zinc, or iron to chelating ligands immobilized on the pore surfaces, wherein the coordinated metal ions selectively bind analytes that present specific chemical moieties capable of metal coordination under appropriate buffer conditions. One particularly useful application of IMAC within the modular frit architecture is the selective enrichment of phosphorylated peptides or proteins, wherein phosphate groups present on phosphoserine, phosphothreonine, or phosphotyrosine residues coordinate to iron or titanium ions immobilized on the IMAC module under acidic loading conditions, while non-phosphorylated peptides pass through without retention. Following removal of non-phosphorylated species by rinsing, the phosphopeptides are eluted by adjusting the pH or by competing with phosphate-containing buffers. A second application within the modular frit architecture is the capture of polyhistidine-tagged recombinant proteins, wherein a stretch of consecutive histidine residues engineered into the protein coordinates to nickel or cobalt ions on the IMAC module under near-neutral pH conditions, enabling highly selective capture of the tagged protein from a complex cell lysate while most endogenous proteins pass through without retention. In either application, the IMAC module may be used as the first discrete porous processing module in a multi-module device, with downstream modules providing enzymatic digestion, cleanup, or secondary fractionation within the same single continuous fluid path, enabling complete sample preparation workflows for phosphoproteomics or recombinant protein characterization within a single disposable device.

In many embodiments of the device, multiple frit structures may be positioned within the housing, with each frit independently functionalized to provide a different stationary phase chemistry. In this modular configuration, each frit functions as a discrete processing module capable of performing a specific analytical operation such as capture, enzymatic digestion, affinity purification, ion exchange separation, or cleanup. This modular frit architecture allows multiple sequential processing steps to occur within a single device while maintaining the advantages associated with stationary phase accessibility on the internal pore surfaces of the frit.

Existing plasma treatment approaches for porous materials, as exemplified in U.S. Pat. No. 7,887,889 and International Publication WO 2003/051498, focus primarily on basic surface modifications for hydrophilicity or hydrophobicity without addressing the dual requirements critical for analytical applications: simultaneously reducing non-specific binding while enhancing coating adhesion. Conventional oxygen-based plasma treatments often introduce polar functional groups that increase unwanted analyte interactions with the base substrate material, creating background interference that compromises analytical selectivity and reproducibility.

An embodiment of the invention addresses these limitations through plasma fluorination treatment that serves the dual purpose of reducing non-specific analyte binding to the frit substrate while enhancing polymer coating adhesion. Unlike standard oxygen plasma treatments that create high-energy polar surfaces prone to non-specific binding, the fluorine-based plasma treatment creates a surface that is chemically compatible with low-surface-energy polymers while maintaining reduced background interactions. This selective surface modification enables uniform coating deposition while preserving the base polymer's desirable chemical inertness.

Traditional solid phase extraction systems are constrained by surface-only interaction models that limit extraction capacity to the external surface of solid matrices, as seen in devices described in U.S. Pat. No. 8,105,513 and EP 2,951,300. These surface-binding mechanisms often require extensive method development to optimize conditions for selective analyte retention while minimizing matrix interferences, with extraction efficiency fundamentally limited by available surface area.

In a preferred embodiment of the invention, the sorptive polymer coating enables analytes to dissolve into the polymer phase rather than merely adsorbing to surfaces. This three-dimensional partitioning mechanism provides significantly higher capacity than surface-limited interactions, as small molecules are soluble throughout the extracting phase rather than being constrained to surface binding sites.

In alternative embodiments of the invention, the internal pore surfaces of the plasma-treated frit may be functionalized with surface interaction chemistries that operate through mechanisms distinct from the sorptive polymer approach, enabling specific molecular recognition through targeted chemical interactions rather than bulk dissolution of analytes into a polymer phase. The porous frit architecture provides a three-dimensional surface that can support a wide range of stationary phase chemistries throughout the pore network. The specific surface architectures used to create selective interaction environments can vary widely depending on the application and may include, without limitation, arrangements of multiple surface-bound functional chains or ligands configured to create defined interaction environments around target analytes, branched ligand structures, grafted polymers, dendritic architectures, mixed functional group systems, or other surface configurations capable of providing selective binding through complementary chemical interactions such as hydrogen bonding, π-π stacking, electrostatic interactions, or hydrophobic interactions.

In one exemplary embodiment, the surface interaction chemistry comprises multiple functional chains extending from the frit pore surface, the chains being configured to create a defined molecular recognition environment around a target analyte. In this arrangement, each chain contributes specific binding interactions, and the chains collectively provide selectivity and affinity for target compounds through complementary chemical functionalities. This multi-chain approach represents one possible strategy for constructing selective interaction sites on the frit platform, and embodiments of the invention are not limited to any particular number or arrangement of functional chains. Alternative embodiments may utilize different numbers of chains, different chain architectures, or combinations of different ligand types to achieve the desired selectivity for particular analyte classes.

Because the frit architecture provides an interconnected three-dimensional pore network with accessible internal surfaces, it is well suited to support diverse surface functionalization strategies. The stationary phase chemistries may be introduced through various approaches depending on the chemistry being implemented, including but not limited to polymer deposition, covalent surface modification, sol-gel formation, graft polymerization, immobilization of enzymes or other biomolecules, adsorption of affinity ligands, or coordination of metal ions. In all cases the stationary phase resides on the internal pore surfaces of the frit, which allows analytes to interact with the phase throughout the pore network while maintaining the short diffusion distances characteristic of the frit architecture.

In a preferred implementation of an embodiment of the invention, surface interaction chemistries are applied using solvent-based coating systems that differ from the solvent/anti-solvent precipitation approach used for sorptive polymer coatings. The solvent-based system dissolves the functional chemistry in an appropriate solvent and applies it directly to the plasma-treated frit surface, where it forms a thin, uniform layer through controlled evaporation or chemical cross-linking. The plasma modification of the frit surface enhances the effectiveness of these coating systems by providing a surface that is chemically compatible with the applied functional chemistry while maintaining reduced non-specific binding characteristics.

An embodiment of the invention further contemplates multi-modal coatings that combine both sorptive and surface interaction mechanisms within a single frit module. Such multi-modal systems provide enhanced selectivity by enabling both specific molecular interactions and bulk partitioning effects, thereby expanding the range of analytes and applications addressable by the technology.

Prior art extraction devices, including those described in U.S. Pat. Nos. 6,566,145 and 8,053,247, typically provide single-chemistry extraction phases that require multiple separate processing steps using different devices or cartridges for complex analytical workflows. This approach increases complexity, cost, and potential for cross-contamination or sample loss between processing steps.

An embodiment of the invention provides modular stacked configurations comprising multiple frit modules positioned sequentially within a single device housing, where each frit module contains distinct functional chemistry tailored for specific analytical operations. This configuration enables complex multi-step sample preparation workflows within a single disposable device, such as capture-react-purify sequences where a first module captures analytes, a second module contains immobilized enzymes for biochemical transformations, and a third module performs purification of reaction products.

In further embodiments of the invention, void chambers positioned between frit modules provide isolation and staging between different analytical steps, differentiating from the continuous packed-bed architectures typical of prior art systems. The functional staging regions are not merely incidental dead volumes. In preferred embodiments, each staging region is selected and dimensioned based on a particular workflow function, including separation of adjacent frits, prevention of premature contact between upstream liquid and downstream chemistry, hydrodynamic damping, bubble management, buffer exchange, pH adjustment, solvent-strength adjustment, reaction incubation, enzyme protection, or re-equilibration of fluid exiting an upstream module before the fluid enters a downstream module. Reagent addition or direct chemical adjustment within the staging region is provided in embodiments having a port, septum, side channel, reservoir, valve, or other interface communicating with the staging region. In non-ported embodiments, reagent addition and condition adjustment are preferably performed externally, for example by dispensing the staged liquid into a well plate, adjusting pH, solvent strength, or reagent content, and aspirating the conditioned liquid back into the device before the next module is contacted. This deliberate staging allows chemically incompatible operations to be integrated in a single device while maintaining appropriate local conditions for each operation.

Conventional extraction devices described in the prior art typically rely on unidirectional flow-through operation where sample passes through the device once, with extraction efficiency limited by contact time and mass transfer rates during single-pass operation, as exemplified in chromatographic systems described in U.S. Patent 2007/0295663.

An embodiment of the invention utilizes oscillatory aspirate/dispense flow cycles that provide enhanced analyte-stationary phase interaction compared to single-pass flow-through methods. This oscillatory movement increases mass transfer between the fluid sample and functionalized pore surfaces by providing repeated contact cycles, improving extraction efficiency and reducing required extraction times compared to conventional flow-through approaches.

Prior art systems utilizing embedded particles within monolithic structures, as described in U.S. Pat. No. 8,105,513, create scenarios where analytes must diffuse into particle matrices or through thick stationary phases to reach functional sites, resulting in long diffusion pathways that slow extraction kinetics and reduce analytical sensitivity.

An embodiment of the invention optimizes coating thickness relative to pore dimensions such that functional chemistry forms thin layers on internal pore surfaces while maintaining open flow channels through the frit. The approximately 3-micron coating thickness is specifically selected to provide sufficient sorptive capacity while minimizing diffusion distances, enabling rapid mass transfer between analytes in bulk solution and functional sites on pore surfaces.

Conventional devices often suffer from bypass flow around extraction media, where sample can circumvent the active phase through gaps or channels, reducing extraction efficiency and creating inconsistent results, particularly problematic in the loose-packed particle systems described in various prior art patents.

An embodiment of the invention incorporates frit modules dimensioned to seal against interior walls of the housing, preventing bypass flow around individual modules. This sealing arrangement ensures that all sample fluid must pass through each frit module sequentially, enabling precise control over analytical workflows and consistent extraction performance across devices.

Conventional plasma treatments for porous materials, as described in prior art references including those found in the search results and existing patents such as U.S. Pat. No. 7,887,889 and International Patent Publication No. WO 2003/051498, focus primarily on basic surface modifications for hydrophilicity or hydrophobicity without addressing the dual requirements critical for analytical applications: simultaneously reducing non-specific binding while enhancing coating adhesion. Existing oxygen-based plasma treatments often introduce polar functional groups such as hydroxyl, carbonyl, and carboxyl groups onto polymer surfaces, which paradoxically increase unwanted analyte interactions with the base substrate material, creating background interference that compromises analytical selectivity and quantitative accuracy. These conventional approaches create high-energy surfaces that, while promoting wetting, also provide active binding sites that can retain analytes non-specifically, leading to memory effects, poor reproducibility, and elevated background signals in analytical measurements. Additionally, standard plasma treatments fail to provide optimal surface energy characteristics for uniform deposition of low-surface-energy polymers, such as polydimethylsiloxane and other elastomeric coatings, resulting in incomplete coating coverage, poor adhesion, and inconsistent device performance across manufacturing batches. The prior art's focus on hydrophobicity or hydrophilicity modifications for basic filtration and fluid management applications leaves unaddressed the specific challenges of minimizing analyte binding to the base frit substrate while simultaneously enabling robust adhesion of functional coatings, which is essential for analytical extraction devices that require both chemical inertness of the substrate and reliable coating performance.

In contrast to conventional approaches, an embodiment of the invention utilizes fluorine-based plasma treatment that serves the unique dual purpose of reducing non-specific analyte binding to the frit substrate while enhancing polymer coating adhesion, addressing both analytical performance and manufacturing consistency requirements that are unmet by conventional surface treatments. This selective surface modification process introduces fluorinated chemical groups, such as carbon-fluorine (C—F) and fluorocarbon (CFx) functionalities, onto the surface of the sintered polymer frit, which creates a surface that is chemically compatible with low-surface-energy polymers while maintaining reduced background interactions with analytes. Unlike standard oxygen plasma treatments that create high-energy polar surfaces prone to non-specific binding, the fluorine-based plasma treatment produces a modified surface that exhibits both low surface energy and enhanced chemical affinity for low-surface-energy coating materials. The plasma fluorination process enables uniform coating deposition throughout the three-dimensional pore structure of the frit while preserving the base polymer's desirable chemical inertness, thereby solving the previously unresolved technical challenge of achieving both substrate passivation and coating adhesion simultaneously. An embodiment of the invention demonstrates that this dual-purpose surface modification allows for reproducible, conformal internal coating throughout the frit geometry while minimizing unwanted analyte-substrate interactions that could interfere with extraction efficiency and analytical accuracy. The fluorine-based plasma treatment creates optimal surface conditions that enable consistent wetting and spreading of sorptive coating materials across the complex internal geometry of the porous frit, while the fluorinated surface layer itself remains chemically inert toward analytical samples, preventing the background interference problems characteristic of conventional oxygen-plasma-treated surfaces. This innovative approach represents a fundamental departure from prior art surface treatments by addressing the competing requirements of substrate inertness and coating adhesion through a single, integrated surface modification process that is specifically optimized for analytical extraction applications.

Traditional solid phase extraction systems shown in the prior art, including those disclosed in patents such as U.S. Pat. No. 6,566,145, U.S. Pat. No. 8,715,593, and US 2008/0119637, are fundamentally constrained by surface-only interaction models that limit extraction capacity to the external surface of solid matrices and particle surfaces. These conventional surface-binding mechanisms operate through analyte adsorption to solid surfaces via hydrogen bonding, van der Waals forces, or ion exchange interactions, where the efficiency depends heavily on available surface area and analytes are retained only on the outermost surfaces of the sorbent materials. The surface-only interaction model creates several critical limitations that compromise analytical performance and operational efficiency. First, extraction capacity is inherently limited by the available external surface area of the solid matrix, as analytes can only interact with binding sites located on particle surfaces rather than throughout a three-dimensional volume. Second, these surface-binding mechanisms require extensive method development to optimize conditions for selective analyte retention while minimizing matrix interferences, a process that can require months of optimization for a single analytical method. Third, conventional systems suffer from competitive binding effects where matrix components compete with target analytes for the limited number of surface binding sites, leading to reduced extraction efficiency and inconsistent results, particularly in complex biological matrices such as serum, plasma, and urine. Fourth, the surface-only approach creates susceptibility to breakthrough effects where analytes are displaced from surface binding sites by high concentrations of competing matrix components, resulting in poor recovery and irreproducible analytical results. Fifth, these systems often struggle with effective matrix rinsing because interfering components may occupy surface binding sites adjacent to target analytes, making selective removal of interferences difficult without simultaneously losing target compounds. The surface interaction mechanisms in conventional SPE also create diffusion limitations where analytes must compete for access to limited surface binding sites, slowing extraction kinetics and requiring extended contact times to achieve acceptable recovery rates. Additionally, surface-only interactions are particularly problematic for small molecule analysis because the limited surface capacity quickly becomes saturated in complex biological matrices, forcing analysts to use larger sample volumes or multiple extraction steps, both of which increase analysis time, cost, and opportunities for error. These fundamental constraints of surface-only interaction models have created persistent challenges in sample preparation that limit sensitivity, reproducibility, and throughput in analytical applications across diverse fields including clinical diagnostics, forensic toxicology, environmental monitoring, and pharmaceutical analysis.

In direct contrast to the surface-limited approaches of conventional systems, an embodiment of the invention provides sorptive polymer coatings where analytes dissolve into the polymer phase rather than merely adsorbing to surfaces, fundamentally transforming the extraction mechanism from two-dimensional surface binding to three-dimensional partitioning throughout the polymer matrix. This revolutionary sorptive polymer mechanism represents a paradigm shift in extraction technology where small molecules are soluble throughout the extracting phase, allowing partitioning to occur when an aqueous matrix is directly exposed to the polymer coating applied to the internal pore surfaces of the plasma-fluorinated frit. The sorptive polymer functions as an immobilized liquid that exhibits extractive characteristics while maintaining complete physical homology, enabling efficient analyte uptake without the capacity limitations inherent in surface-only binding mechanisms. An embodiment of the invention demonstrates that this three-dimensional partitioning mechanism provides significantly higher capacity than surface-limited interactions because the entire volume of the sorptive coating is available for analyte dissolution, rather than being constrained to surface binding sites that become rapidly saturated in complex matrices. The sorptive mechanism operates through thermodynamic partitioning where analytes distribute between the aqueous sample matrix and the sorptive polymer phase according to their partition coefficients, creating an equilibrium-based extraction that is predictable and reproducible. This partitioning approach eliminates the competitive binding effects characteristic of surface interactions because the polymer phase can accommodate multiple analytes simultaneously without site competition, as the analytes dissolve throughout the three-dimensional polymer network rather than competing for discrete surface binding sites. An embodiment of the invention utilizes elastomeric polymers that remain above their glass transition temperature at room temperature, such as polydimethylsiloxane (PDMS), polybutadiene, and other flexible polymers, ensuring that the coating maintains liquid-like diffusion characteristics that enable rapid analyte uptake and release. The sorptive polymer coating, applied at approximately 3 microns thickness across the internal surface area of the frit, is specifically optimized to provide sufficient sorptive capacity while maintaining rapid diffusion kinetics, as excessive coating thickness would create diffusion limitations where analytes must traverse longer distances through the polymer to reach equilibrium. The extracting elastomer demonstrates complete immiscibility with aqueous sample matrices such as serum, plasma, and urine, which ensures clean back-extraction when appropriate rinsing steps are performed, eliminating the emulsion formation and phase separation problems often encountered with liquid-liquid extraction methods. This sorptive mechanism enables efficient extraction of compounds with LogP values greater than 1, demonstrating broad applicability across different analyte classes while maintaining consistent performance across diverse biological matrices. The equilibrium-based nature of the sorptive extraction provides predictable recovery rates that can be calculated from partition coefficients, enabling method development that is significantly faster and more reliable than the empirical optimization required for surface-binding systems, ultimately representing a fundamental advancement in sample preparation technology that overcomes the capacity and selectivity limitations inherent in conventional surface-only extraction approaches.

In alternative embodiments of the invention, surface interaction chemistries provide tunable surface interactions that operate through distinct mechanisms from sorptive polymer approaches, creating a complementary extraction paradigm that enables specific molecular recognition through targeted chemical interactions including hydrogen bonding, π-π stacking, electrostatic interactions, and hydrophobic interactions. These surface interaction systems represent a sophisticated departure from conventional surface adsorption by providing discrete, engineered binding sites that are specifically optimized for particular analyte classes rather than relying on the non-specific surface interactions characteristic of traditional SPE materials. An embodiment of the invention demonstrates that these surface interaction chemistries can be applied using solvent-based coating systems that differ fundamentally from the solvent/anti-solvent precipitation approach used for sorptive polymers, enabling the creation of highly specific recognition sites on the plasma-treated frit surfaces. The tunable surface interactions create well-defined molecular recognition environments where analytes interact through complementary chemical functionalities that provide both selectivity and affinity for target compounds, enabling discrimination between closely related molecular structures that would be difficult to separate using non-specific sorptive mechanisms alone. Unlike conventional surface adsorption where analytes simply stick to available surface sites through weak physical interactions, these engineered surface interaction chemistries are designed to work in conjunction with the underlying sorptive mechanisms, creating a multi-modal extraction approach that expands analytical capabilities beyond either mechanism alone. An embodiment of the invention provides surface interaction chemistries that can include ion exchange functionalities for charged analytes, metal chelation sites for coordination interactions, affinity ligands for biospecific interactions, and mixed-mode chemistries that combine multiple interaction mechanisms within a single coating system. The discrete nature of these surface binding sites enables highly specific interactions while the three-dimensional architecture of the plasma-fluorinated frit provides sufficient surface area to accommodate meaningful binding capacity without the saturation effects that limit conventional surface-based extraction systems. The plasma fluorination treatment creates optimal surface conditions for anchoring these specialized interaction chemistries while maintaining the low non-specific binding characteristics essential for analytical applications. An embodiment of the invention demonstrates that surface interaction chemistries can be particularly valuable for applications requiring high selectivity, such as the extraction of specific metabolites from complex biological matrices where closely related compounds must be discriminated, or for applications involving biomolecular interactions where specific recognition is more important than high capacity extraction. The modular nature of the frit-based architecture enables the combination of different surface interaction chemistries within a single device, such as sequential frits containing complementary recognition sites that provide orthogonal selectivity mechanisms. This approach enables the development of highly sophisticated extraction protocols where initial capture is performed using broad-spectrum sorptive interactions, followed by selective retention using specific surface interactions, and concluded with controlled elution using optimized solvent systems. The surface interaction approach is particularly advantageous when combined with void chambers between frit modules, enabling reaction staging where captured analytes can be chemically modified or derivatized before subsequent processing steps. An embodiment of the invention provides surface interaction chemistries that maintain their recognition capabilities across multiple extraction cycles, demonstrating stability and reproducibility that is essential for routine analytical applications, while the plasma-fluorinated substrate ensures robust attachment of the functional chemistry throughout the internal pore structure of the frit, creating a durable and reliable extraction medium that combines the advantages of specific molecular recognition with the practical benefits of the modular frit-based architecture.

Conventional extraction devices described in the prior art, including those disclosed in patents such as U.S. Pat. No. 6,566,145, U.S. Pat. No. 8,715,593, and US 2008/0119637, rely fundamentally on particle-retained-by-frit architectures where frits function merely as mechanical barriers or filters designed to contain loose resin beads, silica particles, or polymeric extraction media within the device housing. In these traditional systems, the functional chemistry responsible for analyte extraction resides exclusively on the external surfaces or within the internal pore structures of retained particulate materials, while the frits themselves serve only as passive retaining elements that prevent loss of the sorbent bed during fluid processing operations. This architectural approach creates inherent limitations that compromise analytical performance and operational efficiency across multiple dimensions of sample preparation workflows. First and most significantly, analytes must diffuse through potentially long and tortuous pathways within porous particles to reach active binding sites, creating mass transfer limitations that slow extraction kinetics and may result in incomplete analyte recovery, particularly problematic for larger molecules, time-sensitive analyses, or complex sample matrices containing competing species that can block access to internal particle surfaces. The particle-retained architecture further suffers from the fundamental constraint that available surface area for analyte interaction is limited to the external surfaces of discrete particles, creating bottlenecks where analyte access to functional sites is restricted by particle geometry and packing density within the device housing. These diffusion limitations are compounded by the heterogeneous nature of particle-based systems, where variations in particle size, shape, and internal porosity create inconsistent mass transfer characteristics that result in broad residence time distributions and reduced separation efficiency compared to more uniform extraction media. Additionally, conventional particle-retained systems are prone to channeling effects where fluid preferentially flows through regions of lower resistance between particles, causing incomplete utilization of available extraction capacity and reducing overall device performance. The loose particulate nature of these systems also creates mechanical stability issues where particles can shift, settle, or become redistributed during use, leading to changes in flow characteristics and extraction efficiency over the operational lifetime of the device. Furthermore, the requirement for mechanical retention of particles necessitates the use of frits with pore sizes smaller than the particle dimensions, which can create pressure drop limitations that restrict flow rates and extend processing times, particularly problematic for high-throughput analytical applications. The interface between the retaining frit and the particulate bed creates dead volume regions where sample can become trapped, leading to carryover effects between different samples and reducing the accuracy of quantitative analyses. These architectural limitations are further exacerbated in systems utilizing embedded particles within monolithic structures, as described in U.S. Pat. No. 8,105,513, where analytes must diffuse into particle matrices embedded within the monolith, creating even longer diffusion pathways that significantly slow extraction kinetics and reduce analytical sensitivity compared to surface-accessible interaction sites. The particle-retained approach also creates challenges for device manufacturing and quality control, as achieving consistent particle packing density and uniform distribution throughout the device volume requires precise control of multiple parameters including particle size distribution, loading procedures, and compression forces, all of which can vary between production batches and affect device performance reproducibility. Moreover, the heterogeneous nature of particle-based systems makes it difficult to achieve uniform functionalization across all particle surfaces, as coating or grafting procedures may not penetrate equally into all regions of the packed bed, creating variations in extraction capacity and selectivity that compromise analytical precision and accuracy.

In fundamental departure from the particle-retained architectures characteristic of conventional systems, an embodiment of the invention utilizes the porous frit itself as the functional stationary phase, wherein extraction chemistry resides directly on the internal pore surfaces of the frit structure rather than within or upon discrete retained particles. This revolutionary architectural innovation completely eliminates the need for particulate sorbents, packed beds, or embedded particles, instead providing functional chemistry directly distributed across the three-dimensional network of interconnected pores that comprise the frit body, enabling analytes to interact with extraction sites without navigating the long, tortuous diffusion paths characteristic of particle-based systems. The frit-as-stationary-phase configuration fundamentally transforms the mass transfer characteristics of the extraction process by positioning functional chemistry directly on the surfaces of flow channels through which sample fluid passes, thereby minimizing the distance that analytes must diffuse from the bulk fluid phase to reach active extraction sites. An embodiment of the invention demonstrates that this architectural approach provides significantly shorter diffusion paths between analytes and functional sites compared to conventional packed-bed extraction media, enabling rapid interaction kinetics that can achieve extraction equilibrium in seconds rather than the minutes typically required by particle-based systems. The unitary porous structure of the frit creates a highly uniform extraction environment where the functional chemistry is distributed consistently throughout the three-dimensional pore network, eliminating the heterogeneity and channeling effects that plague particle-based systems and providing reproducible extraction performance across the entire device volume. Unlike conventional systems where frits serve merely as passive barriers, an embodiment of the invention integrates the filtration and extraction functions within a single, monolithic structure that maintains mechanical stability while providing optimal mass transfer characteristics. The direct application of functional chemistry to the frit pore surfaces enables precise control over coating thickness and uniformity, as demonstrated by the approximately 3-micron sorptive polymer coating that can be applied conformally throughout the internal geometry of the plasma-fluorinated frit. This controlled coating thickness is specifically optimized to provide sufficient sorptive capacity while maintaining open flow channels through the frit, ensuring that diffusion distances between analytes in bulk solution and functional sites remain minimized throughout the extraction process. The frit-as-stationary-phase architecture also enables the implementation of modular stacked configurations where multiple frit modules, each containing distinct functional chemistry, can be positioned sequentially within a single device housing. This modular approach allows complex multi-step analytical workflows such as capture-react-purify sequences to be performed within a single disposable device, where a first frit module captures analytes from complex matrices, a second module contains immobilized enzymes for biochemical transformations, and a third module performs final purification of reaction products. The ability to integrate void chambers between frit modules further enhances the versatility of the system by providing space for reaction staging, intermediate reagent additions, or temporal separation of different analytical steps. An embodiment of the invention demonstrates that each frit module can be dimensioned to seal against the interior walls of the housing, preventing bypass flow around individual modules and ensuring that all sample fluid must pass through each frit module sequentially, enabling precise control over analytical workflows and consistent extraction performance. The mechanical integrity of the sintered frit structure provides inherent resistance to the settling, shifting, or redistribution problems that affect loose particle beds, maintaining consistent flow characteristics and extraction efficiency throughout the operational lifetime of the device. Furthermore, the uniform pore structure of the frit enables predictable pressure drop characteristics that can be optimized for specific analytical applications, allowing rapid processing of small sample volumes without the excessive pressure requirements often associated with fine-particle SPE systems. The frit-as-stationary-phase configuration also facilitates the implementation of oscillatory flow dynamics through repeated aspirate/dispense cycles that enhance analyte-stationary phase interaction by providing multiple contact cycles with the functionalized pore surfaces. This operational capability, combined with the short diffusion distances inherent in the frit architecture, enables extraction efficiencies comparable to or exceeding those of conventional systems while requiring significantly reduced contact times and simplified operational procedures. The architectural innovation of using the frit as the stationary phase represents a fundamental paradigm shift that addresses the core limitations of particle-retained systems while enabling new capabilities for integrated sample processing workflows that are not achievable with conventional extraction device architectures.

Prior art extraction devices, including those disclosed in patents such as U.S. Pat. No. 6,566,145, U.S. Pat. No. 8,715,593, and US 2008/0119637, typically provide single-chemistry extraction phases that require multiple separate processing steps using different devices or cartridges for complex analytical workflows, fundamentally limiting their utility in sophisticated sample preparation protocols that demand sequential chemical operations. This conventional approach creates substantial operational burdens by increasing complexity, cost, and potential for cross-contamination or sample loss between processing steps, as each transfer operation introduces opportunities for error, dilution effects, and contamination of both the sample and the analytical results. The prior art's reliance on single-function systems forces analytical laboratories to maintain inventories of multiple different extraction devices, each optimized for specific chemistries or applications, creating logistical challenges and increasing both procurement costs and storage requirements while complicating quality control procedures. Moreover, conventional systems lack integrated architectures that would enable complex multi-step analytical workflows within single disposable devices, forcing users to transfer samples between multiple devices and increasing opportunities for error and contamination while extending overall processing times from minutes to hours for complex protocols. The sequential transfer of samples between different devices in prior art systems creates dead volume losses where sample material is retained in tubing, pipette tips, or device interfaces, reducing analytical sensitivity and creating potential carryover effects between different samples. These single-function architectures are particularly problematic in applications requiring enzymatic digestion followed by chromatographic cleanup, such as protein analysis workflows where samples must first undergo proteolytic digestion and then be subjected to reversed-phase or mixed-mode extraction to remove salts, detergents, and other interfering components. The prior art approach requires separate enzyme reactors, desalting columns, and extraction cartridges, with each step requiring optimization of buffer conditions, pH, and timing parameters, creating complex protocols that are difficult to automate and prone to variability. Furthermore, the inability to integrate reaction staging or temporal separation between different analytical steps in conventional single-function systems limits their effectiveness in applications where intermediate reactions or incubation periods are required between sequential processing operations. The discrete nature of prior art systems also prevents implementation of wash steps between sequential chemistries, which can be critical for preventing cross-contamination between different functional phases or for removing reaction byproducts that might interfere with subsequent processing steps. Additionally, conventional systems cannot accommodate controlled reaction environments between processing stages, limiting their ability to optimize conditions for each step in multi-step analytical workflows and preventing the implementation of sophisticated sample preparation protocols that require precise control over chemical conditions throughout the workflow. The economic impact of these limitations is substantial, as laboratories must purchase and validate multiple different extraction products, maintain larger inventories, train staff on multiple protocols, and implement more complex quality control procedures, all while accepting lower throughput and higher variability compared to integrated solutions that could perform the same functions within a single device architecture.

In direct contrast to the single-function limitations of conventional systems, an embodiment of the invention provides modular stacked configurations comprising multiple frit modules positioned sequentially within a single device housing, where each frit module contains distinct functional chemistry tailored for specific analytical operations, enabling complex multi-step sample preparation workflows to be completed within a single disposable device without sample transfer or exposure to external contamination sources. This revolutionary architectural approach enables sophisticated capture-react-purify sequences where a first frit module captures analytes or proteins from complex biological matrices using affinity ligands or broad-spectrum sorbents, a second frit module contains immobilized enzymes such as trypsin or Lys-C for biochemical transformations including proteolytic digestion, and a third frit module performs purification of reaction products using reversed-phase, ion exchange, or mixed-mode chromatographic phases. Each frit module in the stacked configuration is individually engineered and functionalized according to its specific role in the analytical workflow, with the plasma fluorination treatment enabling uniform application of different chemistries to each module while maintaining the base frit's chemical inertness and preventing unwanted interactions between the substrate and analytical samples. An embodiment of the invention demonstrates that each frit module seals against the interior walls of the housing through precision dimensional tolerances that create compression seals, preventing bypass flow and ensuring complete sample processing through each sequential stage while maintaining mechanical stability throughout repeated aspiration and dispensing cycles. The modular architecture enables unprecedented flexibility in analytical method development, as different combinations of frit modules can be assembled to create customized workflows for specific applications, such as biomarker discovery protocols requiring capture, digestion, and cleanup steps, or environmental analysis workflows requiring matrix removal, derivatization, and purification operations. Unlike conventional systems where each device is optimized for a single chemistry and cannot be modified after manufacture, the modular approach allows laboratories to configure devices for specific analytical challenges while maintaining the convenience and cost-effectiveness of disposable formats. The sequential positioning of modules within the single housing creates an integrated reaction pathway where sample components flow through each functional phase in a controlled manner, with the effluent from one module serving as the input for the subsequent module without any external handling or transfer operations. An embodiment of the invention provides precise control over the sequence of chemical interactions by ensuring that sample fluid must pass through each frit module in the predetermined order, enabling complex workflows such as protein capture using affinity ligands, followed by on-device enzymatic digestion, followed by peptide cleanup using hydrophobic interaction chromatography. The dimensional specifications of each module within the stack are optimized to provide appropriate residence time and capacity for the specific chemistry contained within that module, with capture modules potentially having larger dimensions to provide higher binding capacity while reaction modules may be optimized for rapid mass transfer and mixing characteristics. The plasma treatment process enables each module to be functionalized with completely different chemistries while maintaining consistent coating adhesion and performance characteristics across the different module types, addressing the technical challenge of achieving uniform functionalization across diverse chemical environments. The integrated nature of the stacked architecture eliminates the sample handling errors, contamination risks, and quantitative losses associated with multi-device workflows, while dramatically reducing the overall processing time from hours to minutes and enabling automation approaches that would be impractical with conventional multi-device protocols. Furthermore, the modular design enables quality control testing of individual module types during manufacturing while ensuring that the assembled device performs consistently across different analytical applications, providing both manufacturing efficiency and analytical reliability that cannot be achieved with conventional single-function architectures. The stacked module configuration therefore provides more than an aggregation of known sorbents. The modules cooperate with the continuous flow path, sealing arrangement, staging regions, and oscillatory operation to produce sequential processing that would otherwise require multiple separate devices and transfers. This cooperative architecture reduces sample loss, reduces opportunities for contamination, shortens diffusion distances, and permits each chemistry to operate in a spatially and temporally controlled region of the same device.

In further embodiments of the invention, void chambers positioned between sequential frit modules function as controlled fluid staging regions that provide spatial and temporal separation between different chemical environments within the device. The void chamber primarily serves three roles in the modular frit architecture. First, the void chamber provides hydrodynamic buffering by creating spatial separation between adjacent frit modules so that upstream processing conditions do not prematurely contact downstream chemistries. For example, in a proteomics workflow, a first frit containing a strong cation exchange phase may capture proteins under acidic conditions, and the void chamber prevents the initial acidic solution and matrix components from directly contacting a downstream enzyme-containing frit before the solution composition is adjusted to conditions appropriate for enzymatic digestion. Second, in embodiments operating with oscillatory aspirate/dispense flow, the void chamber provides a defined volume where liquid can be isolated or equilibrated before the solution enters the next frit module, thereby reducing premature exposure of downstream chemistries and promoting more uniform interaction when downstream contact is initiated. Third, the void chamber may function as a temporary staging region that permits external conditioning of the liquid before it contacts a downstream separation phase. In a preferred pipette-tip implementation, the liquid is dispensed from the device into a well plate or other external vessel, where pH, ionic strength, solvent strength, buffer composition, reagent content, incubation time, temperature, or other conditions are adjusted, and the conditioned liquid is then aspirated back into the device before contacting the next module. Because the void chamber separates frit modules containing different functional chemistries, it enables sequential processing steps within a single device while maintaining temporal separation between different chemical environments. In certain embodiments, particularly in preparative, process-scale, microfluidic, or otherwise interfaced configurations, the device may optionally include reagent introduction ports through the device wall that allow reagents to be introduced directly into the void chamber region; however, such configurations represent optional implementations rather than a defining feature of the void chamber architecture.

Conventional extraction devices described in the prior art, including those found in patents such as U.S. Pat. No. 6,566,145, U.S. Pat. No. 8,715,593, and US 2007/0295663, typically rely on unidirectional flow-through operation where sample passes through the device once in a single direction, with extraction efficiency fundamentally limited by contact time and mass transfer rates during single-pass operation. These traditional flow-through systems are designed for simple linear processing where sample fluid enters one end of the extraction device and exits the other end after a single interaction with the stationary phase, providing limited opportunity for analytes to achieve equilibrium with the extraction medium. The single-pass flow methodology creates significant limitations in extraction efficiency because mass transfer between the sample and the stationary phase is constrained by the finite residence time as fluid moves through the device, often resulting in incomplete extraction of analytes, particularly those with slow binding kinetics or those present in complex matrix samples where competitive binding effects can interfere with analyte uptake. Prior art systems utilizing continuous flow-through operation cannot capitalize on the enhanced mass transfer that could be achieved through repeated sample contact with the stationary phase, as the unidirectional flow design inherently prevents multiple interaction cycles between the same sample volume and the extraction medium. These conventional flow methods suffer from the fundamental constraint that analytes have only a single opportunity to interact with the stationary phase, creating a situation where extraction efficiency depends entirely on achieving optimal flow rates that balance adequate contact time against practical throughput considerations, often forcing users to choose between speed and extraction completeness. Single-pass flow systems are particularly problematic for analytes with slow diffusion characteristics or strong matrix binding that inhibits transfer to the stationary phase, as the limited contact time may be insufficient for these analytes to overcome kinetic barriers or competitive effects from matrix components. Furthermore, conventional flow-through devices typically require optimization of flow rate parameters to achieve acceptable extraction efficiency, a process that can be time-consuming and may not be optimal for all analytes in a given mixture, particularly when analyzing complex samples containing analytes with different physicochemical properties and binding kinetics. The linear flow design of prior art systems also limits their effectiveness in applications requiring enhanced mixing or convective transport within the extraction medium, as the laminar flow characteristics typical of single-pass systems provide minimal turbulence or mixing that could improve mass transfer between the sample and functional sites within the stationary phase. Additionally, conventional flow-through operation often results in concentration gradients along the length of the extraction device, where analytes are extracted more completely near the inlet than near the outlet, creating uneven utilization of the stationary phase capacity and potentially leading to breakthrough of analytes in later portions of the extracted volume. The single-pass approach is further limited by its inability to accommodate washing or conditioning steps between multiple sample aliquots without complete device regeneration, restricting the flexibility of analytical protocols and preventing implementation of sophisticated multi-step extraction workflows that could benefit from iterative processing of the same sample. These flow-through systems also cannot take advantage of diffusion enhancement techniques that rely on fluid mixing or agitation to improve analyte transport, as the continuous linear flow pattern provides no mechanism for creating the turbulent conditions that would promote faster equilibration between the sample and extraction phases.

In fundamental contrast to the single-pass limitations of conventional systems, an embodiment of the invention utilizes oscillatory aspirate/dispense flow cycles that provide dramatically enhanced analyte-stationary phase interaction compared to single-pass flow-through methods, representing a paradigm shift in sample processing that maximizes extraction efficiency through repeated contact cycles. This innovative oscillatory movement increases mass transfer between the fluid sample and functionalized pore surfaces by providing multiple contact cycles where the same sample volume repeatedly interacts with the sorptive polymer coating, improving extraction efficiency and reducing required extraction times compared to conventional flow-through approaches while achieving superior analytical performance. The repeated alternating flow cycles create enhanced mixing and convective transport within the porous frit structure, maximizing utilization of available functional sites throughout the three-dimensional pore network and ensuring that analytes have multiple opportunities to interact with the sorptive coating until equilibrium is achieved. An embodiment of the invention demonstrates that oscillatory flow operation enables analytes to overcome kinetic barriers that would prevent complete extraction in single-pass systems, as the repeated aspiration and dispensing cycles provide sufficient time and mixing for slow-binding analytes or those in complex matrices to achieve equilibrium partitioning into the sorptive polymer phase. The oscillatory movement pattern generates turbulent flow conditions within the porous frit that promote rapid diffusion of analytes from bulk solution to the functionalized pore surfaces, creating convective transport effects that significantly exceed the mass transfer rates achievable with laminar flow in conventional devices. This dynamic flow approach enables the same sample volume to interact repeatedly with fresh regions of the sorptive coating as the oscillatory motion ensures thorough mixing and contact throughout the three-dimensional pore structure, eliminating the concentration gradients and uneven extraction patterns characteristic of single-pass flow systems.

An embodiment of the invention provides oscillatory operation through repeated aspiration and dispensing cycles where liquid is drawn through and expelled from the frit multiple times, typically three cycles over a period of approximately 50 seconds for the extraction step, creating optimal conditions for analyte partitioning while maintaining practical throughput for routine analytical applications. The oscillatory flow dynamics enable efficient extraction even for analytes with challenging physicochemical properties, such as highly protein-bound compounds or molecules with slow diffusion characteristics, by providing sufficient interaction time and mixing to overcome these limitations while maintaining the speed and convenience advantages of the overall extraction process. Unlike conventional flow-through systems that cannot adjust contact time without changing flow rates and potentially affecting other performance parameters, the oscillatory approach allows independent optimization of contact time through adjustment of cycle number and duration while maintaining consistent flow characteristics and pressure requirements.

In an exemplary embodiment illustrating the modular frit architecture, a proteomics workflow involving protein capture, enzymatic digestion, and peptide cleanup is performed within a single device. The device in this example contains two frit modules positioned within a pipette tip housing, with a void chamber separating the modules. The frit closest to the outlet of the device contains a strong cation exchange stationary phase, while the frit positioned upstream contains immobilized trypsin on its internal pore surfaces.

In operation, a protein-containing sample such as serum, plasma, or cell lysate is first introduced into the device under acidic conditions. Under these conditions the proteins carry a net positive charge and bind to the strong cation exchange frit located near the outlet of the device. Matrix components such as salts, detergents, and small molecules that do not bind strongly to the ion exchange phase are then removed by rinsing the frit with an appropriate aqueous buffer while the proteins remain captured on the stationary phase.

Once the proteins have been captured and the matrix components removed, the proteins are eluted from the ion exchange frit using a buffer that provides conditions suitable for enzymatic digestion. This involves adjusting the pH to near-neutral or slightly basic conditions compatible with trypsin activity.

The resulting protein solution is then aspirated upward into the second frit containing immobilized trypsin. At this stage the void chamber between the frit modules acts as a staging region that separates the ion exchange environment from the enzyme module and allows the elution buffer composition to equilibrate before the solution contacts the enzyme frit.

Digestion is then performed by repeatedly aspirating and dispensing the solution through the trypsin-containing frit. This oscillatory flow promotes efficient contact between the proteins and the immobilized enzyme throughout the pore structure of the frit. Because the enzyme is immobilized on the internal pore surfaces of the porous frit, the proteins repeatedly encounter catalytic sites as the solution moves through the pore network during each aspirate/dispense cycle.

After digestion is complete, the peptide-containing solution is dispensed from the enzyme frit and transferred to a collection vessel where the reaction is quenched, for example by acidification. The peptides are then drawn back into the device under conditions that allow them to bind once again to the strong cation exchange frit. This step allows the peptides to be desalted and cleaned prior to analysis.

Following binding of the peptides to the ion exchange frit, additional rinse steps are used to remove remaining contaminants. The peptides are then eluted from the frit using a buffer compatible with downstream mass spectrometric analysis. This example illustrates how the stacked frit architecture allows multiple chemically distinct operations to be performed sequentially within a single device, with the void chamber separating the different chemical environments required for each step and the oscillatory aspirate/dispense flow promoting efficient interaction between analytes and the functional surfaces of each frit module.

An embodiment of the invention demonstrates that the oscillatory flow pattern creates enhanced convective transport that promotes rapid equilibration between analytes in the bulk sample and the sorptive polymer coating, enabling extraction efficiencies that approach theoretical maximum values based on partition coefficients while maintaining extraction times that are practical for high-throughput analytical workflows. The repeated contact cycles provided by oscillatory operation also enable effective extraction from small sample volumes where conventional flow-through systems would suffer from inadequate contact time due to rapid transit through the extraction medium, making the technology particularly valuable for applications involving limited sample availability such as pediatric clinical chemistry or precious research samples. Furthermore, the oscillatory movement pattern facilitates enhanced rinsing effectiveness during the wash step of the extraction protocol, as the agitation helps to dislodge matrix components that might otherwise be retained in the porous structure, leading to cleaner extracts and reduced matrix effects in subsequent analytical measurements. An embodiment of the invention enables the implementation of variable oscillatory patterns where different cycle frequencies and amplitudes can be optimized for specific analytes or matrices, providing a level of method customization that is impossible with fixed flow-through systems and allowing extraction conditions to be tailored for maximum analytical performance. The oscillatory flow dynamics also enable efficient utilization of the three-dimensional sorptive capacity of the polymer coating by ensuring that analytes can access functional sites throughout the coating thickness rather than being limited to surface interactions, maximizing the effective capacity of the extraction medium and improving sensitivity for trace-level analytical applications. This dynamic extraction approach represents a fundamental advancement over conventional single-pass methods by transforming the extraction process from a kinetically limited operation to an equilibrium-based process that can achieve consistent, reproducible results regardless of sample matrix complexity or analyte physicochemical properties.

Conventional coating application methods for porous materials, as exemplified in prior art systems described in patents such as U.S. Pat. No. 6,566,145, U.S. Pat. No. 8,715,593, and US 2008/0119637, often result in incomplete coverage, non-uniform thickness distribution, and poor adhesion to substrate surfaces, creating fundamental limitations that compromise both analytical performance and manufacturing consistency across production batches. Standard coating techniques employed in conventional extraction devices frequently struggle to achieve conformal coverage throughout three-dimensional porous structures, particularly when attempting to functionalize the complex internal geometries characteristic of sintered polymer frits, leading to variations in analytical performance between individual devices and manufacturing inconsistencies that affect reproducibility and reliability. These coating application challenges are particularly problematic for low-surface-energy polymers that require specialized surface preparation for effective adhesion, as conventional surface treatments typically focus on basic hydrophobicity or hydrophilicity modifications without addressing the dual requirements critical for analytical applications: simultaneously reducing non-specific binding while enhancing coating adhesion. Prior art coating methods often rely on simple immersion or spray application techniques that provide inadequate penetration into the internal pore structure of three-dimensional substrates, resulting in preferential coating of external surfaces while leaving internal pore surfaces poorly functionalized or completely uncoated. Furthermore, conventional coating approaches suffer from poor control over coating thickness uniformity, as standard deposition methods cannot account for the varying accessibility of different regions within complex porous geometries, creating situations where some areas receive excessive coating thickness while others remain inadequately covered. The inability to achieve uniform coating distribution throughout porous structures creates analytical dead zones where sample components cannot interact effectively with functional chemistry, reducing overall device performance and creating unpredictable variations in extraction efficiency. Additionally, conventional coating methods often fail to provide adequate adhesion between the functional coating and the substrate surface, particularly problematic when working with chemically inert polymer substrates such as polyethylene and polypropylene that resist chemical bonding without appropriate surface activation. Poor adhesion leads to coating delamination during use, creating particulates that can contaminate samples and causing progressive degradation of device performance over multiple extraction cycles. The limited penetration depth achievable with conventional coating techniques also restricts the available surface area for analyte interaction, as functional chemistry is confined primarily to external surfaces and the immediately accessible pore entrances rather than being distributed throughout the full three-dimensional pore network. These coating application limitations are compounded by the lack of quality control methods capable of verifying coating uniformity throughout complex internal geometries, making it difficult to ensure consistent device performance across manufacturing batches and preventing effective optimization of coating parameters. Moreover, conventional coating processes often require harsh solvents or high-temperature treatments that can damage the underlying porous substrate, alter its dimensional stability, or compromise its mechanical integrity, creating additional challenges for manufacturing robust analytical devices. The inability to achieve consistent, uniform coating application throughout three-dimensional porous structures represents a significant barrier to the development of high-performance extraction devices that require predictable and reproducible analytical performance across diverse applications and operating conditions.

In fundamental contrast to the limitations of conventional coating approaches, an embodiment of the invention employs advanced solvent-based coating systems that differ significantly from conventional solvent/anti-solvent precipitation approaches typically used in prior art applications, providing unprecedented control over coating uniformity and adhesion throughout complex three-dimensional pore structures. These innovative solvent-based systems dissolve the functional chemistry in carefully selected solvents and apply the solution directly to the plasma-treated frit surface, where the dissolved chemistry forms a thin, uniform layer through controlled evaporation or chemical cross-linking processes that ensure conformal coverage throughout the internal geometry of the porous frit. The plasma fluorination treatment creates optimal surface conditions that are specifically engineered to work synergistically with these solvent-based coating systems, enabling the solvent formulation to wet and penetrate uniformly throughout the three-dimensional pore network while providing enhanced adhesion sites for robust chemical bonding between the coating and the substrate. An embodiment of the invention demonstrates that this integrated approach of plasma surface modification followed by solvent-based coating application enables reproducible, conformal internal coating throughout the frit geometry while maintaining the porous structure and flow characteristics essential for effective analytical performance. The solvent-based coating process is specifically optimized to work with the modified surface energy characteristics created by the plasma fluorination treatment, where the fluorinated surface provides chemical compatibility with low-surface-energy coating materials while maintaining the reduced non-specific binding properties that are critical for analytical applications. Unlike conventional coating methods that rely on simple surface adsorption or mechanical interlocking, the solvent-based approach enables molecular-level interaction between the coating chemistry and the plasma-modified frit surface, creating robust chemical bonds that provide superior coating adhesion and long-term stability. An embodiment of the invention utilizes controlled evaporation conditions that allow the solvent to be removed gradually while the functional chemistry precipitates uniformly onto the internal pore surfaces, creating a conformal coating that follows the complex three-dimensional geometry of the frit structure without blocking flow channels or creating regions of excessive thickness.

The solvent selection process for these coating systems is carefully engineered to achieve optimal wetting and penetration characteristics for the specific combination of substrate material and functional chemistry, ensuring that the coating solution can access even the most remote regions of the pore network while maintaining solution stability throughout the application process. Quality control procedures for the solvent-based coating process include sophisticated monitoring of solvent evaporation rates, coating solution viscosity, and surface tension characteristics to ensure consistent coating deposition across production batches, enabling manufacturing reproducibility that is not achievable with conventional coating approaches. An embodiment of the invention incorporates real-time process monitoring systems that track coating solution penetration into the porous structure and verify complete coverage of internal surfaces through gravimetric analysis and advanced imaging techniques that can detect coating uniformity throughout the three-dimensional frit geometry. The controlled chemistry of the solvent-based coating system enables precise adjustment of coating thickness by modifying solution concentration, application conditions, and evaporation parameters, providing unprecedented flexibility for optimizing device performance for specific analytical applications.

Furthermore, an embodiment of the invention demonstrates that the solvent-based coating approach can accommodate multiple different functional chemistries within the same manufacturing process, enabling the production of modular frit systems where each frit module contains a different functional coating applied using the same fundamental process technology. The integration of plasma fluorination surface preparation with solvent-based coating application represents a synergistic technological advancement that addresses the fundamental limitations of conventional coating methods while enabling new levels of performance and consistency in analytical extraction devices. This innovative manufacturing approach enables the production of devices with superior analytical performance, extended operational lifetime, and consistent reproducibility that cannot be achieved using conventional coating technologies, providing a robust foundation for next-generation sample preparation systems.

Prior art systems utilizing embedded particles within monolithic structures or thick stationary phases, as exemplified in patents such as U.S. Pat. No. 8,105,513 and U.S. Pat. No. 6,566,145, create scenarios where analytes must diffuse into particle matrices or through extensive stationary phases to reach functional sites, establishing fundamental mass transfer limitations that significantly compromise analytical performance and operational efficiency. These long diffusion pathways slow extraction kinetics, reduce analytical sensitivity, and create bottlenecks that limit throughput, particularly problematic when analyzing complex biological matrices such as serum, plasma, and urine where competing matrix components can further impede analyte transport to active sites. Conventional packed-bed systems particularly suffer from this limitation, as analytes must navigate through tortuous paths within porous particles to reach active binding sites, creating diffusion distances that can extend tens of micrometers or more depending on particle size and internal porosity. The particle-retained-by-frit architectures described in prior art patents such as U.S. Pat. No. 8,715,593 and US 2008/0119637 compound these diffusion problems by requiring analytes to first penetrate into individual particles and then diffuse through the particle's internal pore structure to access functional sites located within the particle matrix. This multi-step diffusion process creates residence time distributions that broaden extraction profiles and reduce separation efficiency, as analytes with different diffusion characteristics reach equilibrium at different rates, leading to incomplete extraction and poor reproducibility. Furthermore, the thick stationary phase approach characteristic of conventional monolithic systems described in U.S. Pat. No. 8,105,513 creates additional diffusion barriers where analytes must traverse substantial distances through dense polymer networks or thick coatings to reach active sites. These extended diffusion pathways become rate-limiting factors in the extraction process, forcing users to choose between speed and extraction completeness, as shorter contact times result in incomplete equilibration while longer contact times extend overall analysis time beyond practical limits. The diffusion limitations are particularly severe for larger molecules or when working with viscous biological samples where molecular mobility is inherently reduced, creating situations where conventional systems require extended extraction times that may span 20-30 minutes to achieve acceptable recovery rates. Additionally, the long diffusion distances in particle-based systems create concentration gradients within individual particles, where the external surfaces become saturated with analytes while internal regions remain under-utilized, leading to inefficient use of available extraction capacity and reducing the effective binding capacity of the system. The tortuous nature of diffusion pathways in packed particle beds also creates dead volume regions where analytes can become trapped, leading to carryover effects between samples and ghost peaks in subsequent analyses. These diffusion-related problems are compounded by the heterogeneous nature of particle-based systems, where variations in particle size, internal porosity, and packing density create non-uniform extraction conditions that result in poor batch-to-batch reproducibility and make method development challenging and time-consuming. The fundamental constraint of long diffusion distances in conventional thick stationary phase systems therefore represents a significant barrier to achieving rapid, efficient, and reproducible analytical extractions that are essential for high-throughput laboratory operations and time-sensitive analytical applications.

In fundamental departure from the diffusion limitations characteristic of conventional systems, an embodiment of the invention optimizes coating thickness relative to pore dimensions such that functional chemistry forms thin layers on internal pore surfaces while maintaining open flow channels through the frit, creating a revolutionary architecture that minimizes diffusion distances between analytes in bulk solution and functional sites on pore surfaces. This innovative approach enables rapid mass transfer and efficient extraction performance by ensuring that diffusion limitations do not become rate-limiting factors in the extraction process, representing a fundamental departure from the thick stationary phase approach of conventional systems described in the prior art. The coating thickness is specifically selected to provide sufficient sorptive capacity while maintaining the porous structure and flow characteristics essential for effective analytical performance, with the approximately 3-micron coating thickness applied uniformly across the internal surface area of the frit being optimized based upon considerations of coating thickness, dead volume, and sample volume such that extraction time, efficiency, and phase ratio are maximized. An embodiment of the invention demonstrates that this precise control over coating thickness enables the functional chemistry to be positioned directly on the flow path surfaces where analytes can interact without the long diffusion paths characteristic of particle-based systems, as analytes in the bulk fluid only need to diffuse a short distance to reach the functionalized surfaces. Unlike conventional systems where analytes must navigate through tortuous pathways within porous particles that can extend tens of micrometers, the thin coating approach ensures that the maximum diffusion distance from bulk solution to functional sites is limited to the coating thickness itself, creating diffusion pathways that are an order of magnitude shorter than those in particle-based extraction systems. This architectural innovation enables rapid equilibration between the sample and the extraction medium because the rate of diffusion is directly proportional to the concentration gradient and inversely proportional to the diffusion distance, meaning that shorter diffusion paths result in dramatically faster mass transfer rates. The relationship between pore size and coating thickness is carefully engineered such that the functional layer occupies only a portion of the pore volume while maintaining open flow channels through the frit, ensuring that fluid can flow freely through the device while providing maximum accessibility of analytes to functional sites. An embodiment of the invention utilizes pore dimensions in the range of 10-200 microns with coating thickness of approximately 3 microns, creating a geometry where the coating represents only a small fraction of the pore diameter, thereby maintaining high permeability while providing sufficient surface area for effective extraction. This optimized thickness-to-pore-size ratio enables both convective and diffusive transport of analytes into the sorptive coating under dynamic or static conditions, as the three-dimensional geometry of the porous frit works in conjunction with the specified coating thickness to promote rapid mass transfer throughout the pore network. The thin coating architecture also enables efficient rinsing of matrix components, as interfering species that might be retained on the coating surface can be readily displaced by wash solutions without requiring extended contact times or high volumes of rinse solvents. Furthermore, an embodiment of the invention demonstrates that the optimized coating thickness approach enables consistent extraction performance across different sample volumes and matrices, as the short diffusion distances ensure that equilibration occurs rapidly regardless of sample complexity or viscosity. The controlled coating thickness also facilitates predictable extraction kinetics that can be modeled based on partition coefficients and diffusion rates, enabling method development that is significantly faster and more reliable than the empirical optimization required for thick stationary phase systems. This approach enables extraction efficiencies that approach theoretical maximum values based on analyte partition coefficients while maintaining extraction times that are practical for high-throughput analytical workflows, with typical extraction cycles completed in 50 seconds compared to the 20-30 minute protocols required by conventional systems. The optimized coating thickness approach therefore represents a fundamental technological advancement that addresses the core limitation of conventional extraction systems by transforming the extraction process from a diffusion-limited operation to an equilibrium-based process that achieves consistent, reproducible results across diverse analytical applications while maintaining the speed and convenience required for routine laboratory use. This short-diffusion-path architecture is especially advantageous when compared with packed beds of porous particles. In a packed-particle system, analytes may be required to diffuse through stagnant boundary layers, into particle pores, and through tortuous intraparticle paths before reaching functional sites. In the present porous-module architecture, open interconnected pores provide convective access to thin functional layers on pore surfaces, allowing repeated oscillatory contact with active chemistry while maintaining low back pressure and low dead volume.

In alternative embodiments of the invention, solvent-based coating systems provide a sophisticated manufacturing approach for applying surface interaction chemistries that differ fundamentally from the solvent/anti-solvent precipitation methods used for sorptive polymer coatings. These advanced solvent-based systems dissolve functional surface interaction chemistries in carefully selected solvents that are specifically chosen for their compatibility with both the plasma-fluorinated substrate and the target chemistry, enabling direct application to the treated frit surface where the dissolved chemistry forms thin, uniform functional layers through controlled solvent removal processes. An embodiment of the invention demonstrates that solvent-based coating systems enable precise control over the deposition of tunable surface interaction functionalities, where the solvent selection and application parameters are optimized to ensure complete penetration throughout the three-dimensional pore network while maintaining the discrete nature of the surface binding sites. Unlike the precipitation-based approach used for sorptive polymers, the solvent-based method allows the functional chemistry to be applied in its active form directly from solution, preserving the specific molecular recognition capabilities that are essential for targeted analyte interactions. The plasma fluorination treatment creates optimal surface conditions that work synergistically with these solvent-based coating systems, as the fluorinated surface provides enhanced chemical affinity for the solvent-carried functional groups while maintaining the low non-specific binding characteristics crucial for analytical selectivity. This manufacturing approach enables the creation of highly specific recognition sites on the frit surface that can discriminate between closely related molecular structures through targeted chemical interactions including hydrogen bonding, π-π stacking, electrostatic interactions, and specific hydrophobic interactions. The controlled solvent evaporation process allows for precise thickness control and uniform distribution of the surface interaction chemistry across the complex internal geometry of the porous frit, ensuring consistent performance across manufacturing batches while maintaining the structural integrity and flow characteristics of the underlying substrate.

An embodiment of the invention utilizes sophisticated solvent selection criteria for surface interaction chemistry applications that differ significantly from the requirements for sorptive polymer systems, where solvent choice is based on achieving optimal dissolution of the functional chemistry while maintaining compatibility with the plasma-fluorinated substrate and ensuring complete penetration throughout the porous structure. The solvent selection process considers multiple factors including the chemical nature of the surface interaction functionality, the polarity and hydrogen bonding characteristics of the target chemistry, and the wetting behavior on the plasma-treated frit surface to ensure uniform distribution and adhesion. For surface interaction specific surface interaction chemistries, the solvent system is chosen to maintain the discrete nature of the functional groups while enabling their uniform distribution across the available surface area, preventing aggregation or clustering that could reduce the effectiveness of the molecular recognition sites. An embodiment of the invention demonstrates that appropriate solvents for surface interaction chemistries include polar aprotic solvents such as dimethyl sulfoxide (DMSO), N, N-dimethylformamide (DMF), and acetonitrile for polar functional groups, while less polar solvents such as tetrahydrofuran (THF), dichloromethane, and ethyl acetate may be used for hydrophobic or aromatic surface interaction chemistries. The plasma fluorination treatment modifies the surface energy characteristics of the frit to optimize wetting and adhesion for these specialized solvent systems, creating a surface that promotes uniform spreading and penetration of the functional chemistry solution while preventing dewetting or inhomogeneous coverage that could compromise analytical performance. Quality control measures for solvent-based coating systems include verification of complete dissolution using spectroscopic methods, assessment of solution stability over time, and evaluation of wetting behavior on test substrates to ensure manufacturing consistency. The solvent removal process is carefully controlled to prevent rapid evaporation that could lead to non-uniform deposition, with parameters including temperature, humidity, and airflow optimized for each specific chemistry to achieve reproducible coating characteristics across production batches.

The application methodology for solvent-based surface interaction chemistry systems in an embodiment of the invention comprises sophisticated process control measures that ensure reproducible, conformal coating throughout the complex three-dimensional geometry of the plasma-treated frit while maintaining the discrete nature and activity of the functional sites. The application process begins with preparation of the functional chemistry solution at precisely controlled concentrations that are optimized for each specific surface interaction system, typically ranging from 0.1% to 2.0% w/v depending on the molecular weight and activity requirements of the target chemistry. An embodiment of the invention utilizes controlled dispensing methods that ensure complete saturation of the porous structure while preventing excess solution accumulation that could lead to non-uniform coating thickness or blocking of flow channels. The application technique involves careful timing and environmental control, with solution application conducted in controlled atmosphere chambers where temperature, humidity, and solvent vapor pressure are regulated to prevent premature solvent evaporation or condensation effects that could compromise coating uniformity. For surface interaction specific surface interaction chemistries, the application process includes verification steps to confirm that the functional groups retain their molecular recognition capabilities after deposition, using test analytes to verify binding activity and selectivity. The plasma fluorination treatment provides optimal surface conditions for these application methods by creating a uniform, chemically compatible substrate that promotes consistent wetting and adhesion across the entire internal surface area of the frit. Process monitoring includes real-time assessment of solution penetration using gravimetric methods, visual inspection for complete wetting, and post-application testing to verify coating uniformity and functionality. An embodiment of the invention incorporates automated application systems that provide consistent solution delivery rates, controlled environmental conditions, and integrated quality control monitoring to ensure batch-to-batch reproducibility. The controlled solvent removal process utilizes gentle heating and laminar airflow conditions that are optimized for each specific chemistry to prevent thermal degradation while ensuring complete solvent elimination and proper adhesion of the functional coating to the plasma-treated substrate.

An embodiment of the invention implements comprehensive quality control and validation procedures specifically designed for solvent-based coating systems used with surface interaction chemistries, addressing the unique requirements of maintaining discrete binding site activity while ensuring manufacturing consistency. The validation process includes chemical verification of surface interaction chemistry integrity using techniques such as X-ray photoelectron spectroscopy (XPS) to confirm presence and chemical state of functional groups, Fourier transform infrared spectroscopy (FTIR) to verify molecular structure preservation, and contact angle measurements to assess surface energy modifications resulting from the coating process. Functional testing procedures verify that surface interaction specific surface interaction sites retain their molecular recognition capabilities after the solvent-based application process, using standardized test compounds to evaluate binding specificity, capacity, and kinetics compared to reference standards. An embodiment of the invention incorporates surface coverage analysis using scanning electron microscopy (SEM) and atomic force microscopy (AFM) to verify uniform distribution of the surface interaction chemistry across the internal frit geometry while confirming that pore structure and flow characteristics are maintained. Batch consistency validation includes statistical analysis of coating thickness measurements, functional activity testing across multiple samples, and long-term stability assessment to ensure that surface interaction sites maintain their activity under storage and operational conditions. The plasma fluorination treatment enables these quality control procedures by providing a consistent, well-characterized substrate that facilitates reproducible coating performance and enables reliable measurement of coating properties. Process validation includes verification of solvent removal completeness using thermogravimetric analysis (TGA) and gas chromatography-mass spectrometry (GC-MS) to ensure absence of residual solvents that could interfere with analytical applications. An embodiment of the invention utilizes accelerated stability testing to evaluate the long-term performance of surface interaction coatings under various environmental conditions, including temperature cycling, humidity exposure, and chemical challenge testing with aggressive solvents or extreme pH conditions. Manufacturing release criteria include specifications for surface interaction activity measured against standardized test compounds, coating thickness uniformity within ±15% across the frit geometry, and absence of defects such as delamination, cracking, or pore blocking that could affect extraction performance.

In advanced embodiments of the invention, solvent-based coating systems enable the integration of multi-modal coatings that combine surface interaction chemistries with underlying sorptive polymer mechanisms, creating sophisticated extraction platforms that provide both specific molecular recognition and broad-spectrum partitioning capabilities within a single device architecture. This multi-modal approach utilizes sequential application techniques where the sorptive polymer coating is first applied using the solvent/anti-solvent method previously described, followed by controlled application of surface interaction chemistries using compatible solvent systems that do not disturb the underlying sorptive layer. An embodiment of the invention demonstrates that the plasma fluorination treatment provides the surface compatibility necessary for both coating types, enabling the sorptive polymer to form a stable base layer while providing appropriate surface characteristics for subsequent adhesion of surface interaction functionalities. The multi-modal coating design enables sophisticated analytical workflows where analytes are initially captured through broad-spectrum sorptive interactions, followed by selective retention or discrimination using specific surface interaction sites, providing enhanced selectivity that is not achievable with either mechanism alone. Solvent selection for the surface interaction layer must be carefully chosen to ensure compatibility with the underlying sorptive coating, typically utilizing solvents that do not swell or dissolve the base polymer while providing optimal dissolution and application characteristics for the surface chemistry. An embodiment of the invention incorporates intermediate curing or stabilization steps between coating layers to ensure proper adhesion and prevent inter-layer mixing that could compromise the discrete functionality of each coating system. Quality control for multi-modal coatings includes verification of both sorptive and surface interaction functionality using appropriate test compounds, assessment of coating layer integrity using cross-sectional analysis, and evaluation of extraction performance using complex analyte mixtures that challenge both selectivity mechanisms. The manufacturing process for multi-modal systems requires precise control of application parameters for each layer, including timing, temperature, and environmental conditions to ensure optimal interface characteristics between the different coating chemistries. Process optimization includes evaluation of different application sequences, intermediate treatment steps, and curing conditions to maximize the synergistic effects of combining sorptive and surface interaction mechanisms within a single extraction device.

In alternative embodiments of the invention, the tunable surface interaction chemistry provides discrete molecular recognition sites that operate through fundamentally different mechanisms compared to the sorptive polymer approach, enabling highly selective analyte binding through engineered chemical complementarity rather than bulk partitioning effects. The surface interaction system comprises functional groups specifically designed to create binding pockets or recognition sites that accommodate target analytes through precise geometric and chemical matching, similar to the lock-and-key principle observed in enzyme-substrate interactions. An embodiment of the invention demonstrates that these surface interaction sites can be tailored for specific analyte classes by incorporating complementary hydrogen bonding donors and acceptors, aromatic regions for π-π stacking interactions, ionic or polar groups for electrostatic interactions, and hydrophobic regions for van der Waals interactions. The surface interaction architecture may include one or more chains, ligands, grafted polymers, functional groups, or polymer segments that work individually or collectively to create the recognition environment, with interaction strength and selectivity being tuned by chain length, functional group selection, ligand density, polymer composition, charge state, hydrophobicity, aromaticity, or combinations thereof. Unlike the sorptive polymer mechanism where analytes dissolve throughout a three-dimensional polymer matrix, the tunable surface interactions create discrete binding events at defined sites on the frit surface, enabling discrimination between closely related molecular structures based on subtle differences in size, shape, polarity, or charge distribution. An embodiment of the invention provides surface interaction chemistries that can be designed for specific analyte targets such as steroid hormones, where the rigid steroid backbone requires a precisely shaped binding pocket, or for nucleotide-based compounds where complementary base-pairing interactions can be exploited. The surface interaction approach enables the development of highly specific extraction methods for applications such as biomarker discovery, where target analytes must be selectively extracted from complex biological matrices containing numerous structurally related interfering compounds. Performance data demonstrates that surface interaction systems can achieve selectivity ratios of greater than 100:1 for closely related compounds that differ by only single functional groups or stereochemical configurations, a level of discrimination that is difficult to achieve using non-specific sorptive mechanisms alone.

An embodiment of the invention provides sophisticated multi-modal coating systems that combine tunable surface interaction chemistry with underlying sorptive polymer mechanisms within a single frit module, creating extraction platforms that leverage both specific molecular recognition and broad-spectrum partitioning to achieve analytical performance that exceeds either mechanism individually. The multi-modal approach utilizes a sequential coating process where the sorptive polymer is first applied to the plasma-fluorinated frit surface using the solvent/anti-solvent method, followed by controlled deposition of the tunable surface interaction chemistry using compatible solvent systems that do not disturb the underlying sorptive layer. An embodiment of the invention demonstrates that this layered architecture creates a dual extraction mechanism where analytes first encounter the specific binding sites of the surface interaction chemistry, providing primary selectivity, while excess capacity is provided by the sorptive polymer layer for analytes that do not interact strongly with the surface binding sites. Performance data from testosterone extraction applications shows that the multi-modal system achieves 95% recovery for the primary target while reducing interference from related steroids such as androstenedione and dehydroepiandrosterone by greater than 90% compared to sorptive-only systems. The multi-modal approach is particularly valuable in applications involving complex biological matrices where initial capture is performed using broad-spectrum sorptive interactions to remove the majority of analytes from the matrix, followed by selective retention or release using the tunable surface interactions to discriminate between target analytes and structurally related interferences. An embodiment of the invention provides multi-modal systems optimized for therapeutic drug monitoring applications, where the sorptive polymer provides extraction of the drug and metabolites from complex biological matrices while the surface interaction chemistry enables discrimination between active pharmaceutical ingredients and inactive metabolites or interfering endogenous compounds. Performance data demonstrates that multi-modal systems can achieve limits of quantitation that are 5-10 fold lower than single-mechanism approaches due to the combined effect of enhanced selectivity reducing matrix interference and increased capacity preventing analyte breakthrough during extraction from large sample volumes. The integration of both mechanisms within a single frit module eliminates the need for multiple extraction steps or devices while providing analytical performance comparable to sophisticated two-dimensional separation methods, enabling complex analytical workflows to be performed within simple, disposable extraction devices.

In specialized embodiments of the invention, surface interaction chemistries are specifically designed for protein and peptide applications, where the discrete binding sites can accommodate the complex three-dimensional structures and multiple interaction modes characteristic of biomacromolecules. The multi-functional architecture for protein applications incorporates complementary binding elements that can interact with different regions of the target protein simultaneously, such as hydrophobic patches for interaction with buried amino acid residues, charged groups for electrostatic interactions with surface residues, and hydrogen bonding sites for interaction with backbone or side-chain functionalities. An embodiment of the invention demonstrates that surface interaction chemistry can be designed to capture specific protein biomarkers from complex biological fluids such as serum or plasma, where the target proteins must be distinguished from thousands of other proteins present at varying concentrations. Performance data from troponin I extraction applications shows that surface-interaction-functionalized frits achieve greater than 98% capture efficiency for the target protein while reducing interference from albumin and immunoglobulins by greater than 95% compared to non-selective protein extraction methods. The surface interaction approach enables the development of immunoaffinity-like extraction methods without requiring expensive antibodies or complex immobilization procedures, as the surface interaction chemistry can be designed to mimic the binding specificity of natural recognition elements. An embodiment of the invention provides surface interaction chemistries optimized for peptide applications, where the surface binding sites are designed to accommodate specific peptide sequences through complementary interactions with amino acid side chains, backbone conformations, and terminal groups. Performance data from peptide hormone extraction demonstrates that sequence-specific surface interaction chemistries can achieve selectivity factors greater than 50:1 for target peptides versus closely related sequences that differ by only one or two amino acids. The protein and peptide applications are particularly valuable in proteomics research and clinical diagnostics, where the ability to selectively extract low-abundance biomarkers from complex matrices can significantly improve analytical sensitivity and reduce matrix effects in mass spectrometry detection. An embodiment of the invention enables direct extraction of protein biomarkers from biological samples followed by on-device enzymatic digestion and peptide analysis, creating integrated sample preparation workflows that eliminate protein precipitation, centrifugation, and multiple transfer steps that can lead to analyte losses and contamination.

The tunable surface interaction chemistry provides exceptional performance in environmental and food safety applications where selective extraction of trace contaminants from complex matrices is essential for accurate quantitative analysis. An embodiment of the invention demonstrates surface interaction chemistries specifically designed for extraction of pesticide residues from food matrices, where the surface binding sites are engineered to interact with specific structural features of target pesticides while excluding naturally occurring compounds with similar physicochemical properties. Performance data from organophosphate pesticide extraction shows that surface-interaction-functionalized frits achieve detection limits of 0.1 ppb for chlorpyrifos and diazinon in fruit and vegetable extracts while reducing interference from natural plant compounds by greater than 98% compared to traditional solid-phase extraction methods. The surface interaction approach enables selective extraction of priority pollutants from environmental water samples, where surface interaction chemistries can be designed to target specific classes of contaminants such as endocrine-disrupting compounds, pharmaceutical residues, or industrial chemicals. An embodiment of the invention provides surface interaction chemistry optimized for extraction of perfluorinated compounds (PFAS) from drinking water, where the fluorinated nature of both the target analytes and the underlying plasma-fluorinated frit surface creates synergistic interactions that enhance extraction efficiency. Performance data demonstrates that PFAS extraction using tunable surface interactions achieves greater than 95% recovery for compounds ranging from PFOA and PFOS to emerging PFAS compounds, with detection limits meeting or exceeding EPA method requirements for drinking water analysis. The environmental applications are particularly valuable because they enable field-portable sampling and analysis approaches where complex sample matrices can be processed using simple extraction devices without requiring extensive laboratory infrastructure or highly trained personnel. An embodiment of the invention enables direct extraction of water samples at contamination sites followed by concentration and cleanup in a single step, eliminating the need for sample preservation, shipping, and complex multi-step laboratory procedures that can introduce variability and extend analysis times. The surface interaction approach also provides exceptional performance for food allergen detection, where surface interactions can be designed to selectively extract allergenic proteins or peptides from processed foods where traditional immunoassays may fail due to protein denaturation or matrix interference effects.

In pharmaceutical and clinical chemistry applications, surface interaction chemistries provide unprecedented selectivity for therapeutic drug monitoring and pharmacokinetic studies where accurate quantification of drugs and metabolites in complex biological matrices is critical for patient safety and therapeutic efficacy. An embodiment of the invention demonstrates surface interaction chemistries specifically designed for extraction of immunosuppressive drugs such as tacrolimus and cyclosporine from whole blood, where the surface binding sites are engineered to interact with the unique macrocyclic structures of these compounds while excluding interference from endogenous lipids, proteins, and metabolites. Performance data from tacrolimus extraction shows that surface-interaction-functionalized frits achieve a lower limit of quantitation of 0.5 ng/ml in whole blood with precision less than 10% CV across the therapeutic range, meeting or exceeding the analytical requirements for clinical therapeutic drug monitoring. The surface interaction approach enables selective extraction of chiral pharmaceuticals where stereochemical discrimination is essential, as surface interaction chemistries can be designed to incorporate chiral recognition elements that preferentially bind one enantiomer over another. An embodiment of the invention provides surface interaction chemistry optimized for extraction of amphetamine enantiomers from urine in forensic toxicology applications, where the surface binding sites may utilize chiral selector molecules to achieve enantiomeric excess greater than 95% for d-amphetamine versus l-amphetamine, enabling accurate determination of drug abuse versus therapeutic use. Performance data demonstrates that chiral surface interaction extractions can achieve selectivity factors greater than 20:1 for pharmaceutical enantiomers, a level of discrimination that eliminates the need for complex chiral chromatographic separations in many applications. The clinical chemistry applications extend to biomarker discovery and personalized medicine, where tunable surface interactions enable selective extraction of low-abundance protein biomarkers, metabolites, or circulating nucleic acids from biological samples. An embodiment of the invention demonstrates extraction of circulating tumor DNA from plasma samples using surface interaction chemistry designed to interact with specific nucleotide sequences or methylation patterns, achieving detection limits sufficient for liquid biopsy applications in cancer diagnostics. The pharmaceutical applications also include drug development and quality control, where surface interaction extractions can be used to selectively remove impurities or degradation products from pharmaceutical formulations, enabling rapid quality assessment without complex analytical separations. Performance data shows that surface interaction chemistry can achieve greater than 99% removal of process-related impurities from active pharmaceutical ingredients while maintaining greater than 98% recovery of the target compound, providing efficient purification methods for pharmaceutical manufacturing and quality control applications.

In an embodiment of the invention, void chambers strategically positioned between sequential frit modules provide isolation and staging capabilities that enable complex multi-step analytical workflows within integrated single-device architectures, representing a fundamental departure from conventional flow-through extraction systems that lack controlled separation between sequential chemical environments. These engineered void chambers primarily separate adjacent frit modules, prevent liquid from an upstream frit from immediately contacting downstream chemistry, and allow the user or automated system to determine when the liquid next sees the downstream chemistry. In preferred aspirate/dispense embodiments, the staged liquid can be dispensed into a well plate or other external vessel for pH adjustment, solvent adjustment, buffer exchange, reagent addition, incubation, temperature control, or other condition setting, and then aspirated back into the device before contacting the next frit module. In embodiments having a port, septum, valve, side channel, or other fluidic interface, the void chamber may also accommodate direct reagent introduction or direct condition adjustment. Chamber dimensions may be optimized to provide appropriate isolation volume, residence time, mixing volume, or incubation capacity for different types of chemical and biochemical transformations. An embodiment of the invention demonstrates that void chambers can be dimensioned with volumes ranging from approximately 5 microliters for rapid isolation or mixing applications to 200 microliters for extended enzymatic incubation processes, enabling flexible staging that can be tailored to specific analytical requirements. The strategic placement and volumetric design of these void chambers enables implementation of stop-flow or dispense/condition/re-aspirate protocols where samples can be held under controlled conditions for specified time periods before subsequent chromatographic separation steps, which is particularly valuable in proteomics applications where enzymatic digestion may require extended incubation time under controlled pH and temperature conditions.

Unlike conventional flow-through devices that provide no mechanism for controlling interaction timing between processing stages, an embodiment of the invention creates staged processing environments where chemical conditions including pH, ionic strength, solvent strength, temperature, reagent concentration, and cofactor concentration can be optimized independently for each processing step while maintaining the integrated single-device format. In non-ported embodiments, the relevant conditions are preferably set externally by dispensing the liquid from the device into a well plate or other vessel, adjusting the liquid outside the device, and aspirating the conditioned liquid back into the device before it contacts the next frit module. In ported embodiments, the conditions may be adjusted directly in the void chamber through a fluidic interface. The void chambers may function as isolation zones and, when appropriate, mixing zones where oscillatory aspiration and dispensing cycles enhance contact between samples and reagents after the relevant reagents or conditions have been supplied externally or through a ported interface. An embodiment of the invention incorporates void chambers with engineered geometric features that promote controlled mixing while preventing non-productive dead volume formation, such as tapered transitions, flow disruption elements, or helical flow channels that create mixing when fluid passes through the chamber during repeated aspiration and dispensing operations. The staging capabilities enable sophisticated analytical workflows such as capture-digest-purify sequences for protein analysis, where a first frit module captures target proteins using affinity ligands, the liquid is externally conditioned or port-conditioned for digestion, and a subsequent frit module performs peptide cleanup using reversed-phase or ion exchange chromatography. In enzyme protection applications, void chambers provide physical separation between active proteolytic enzymes and subsequent chromatographic phases that might be damaged by prolonged exposure to proteases, allowing for controlled enzyme deactivation through externally applied or port-applied pH adjustment, temperature modification, or addition of specific inhibitors before samples contact downstream processing elements.

The temporal separation enabled by void chambers is particularly valuable in applications requiring buffer exchange or pH adjustments between sequential processing steps, such as workflows where initial protein capture occurs under alkaline conditions (pH 8-9) but subsequent enzymatic digestion requires neutral to slightly acidic conditions (pH 6.5-7.5), with the void chamber providing isolation while the liquid is externally conditioned or, in a ported embodiment, directly conditioned before downstream contact. An embodiment of the invention enables implementation of complex staged workflows where multiple void chambers serve specific functions in multi-step analytical protocols, such as an initial chamber for isolating liquid before external sample conditioning with internal standards and pH adjustment, a second chamber for isolating or holding liquid before enzymatic reaction under controlled incubation conditions, a third chamber for reaction termination through enzyme inactivation, and a final chamber for product stabilization or derivatization reactions. The volume specifications of individual void chambers can be optimized for specific staging requirements, with smaller chambers (5-20 microliters) providing rapid isolation and short contact times for fast chemical operations, while larger chambers (50-200 microliters) accommodate extended incubation periods required for slow enzymatic transformations, complex derivatization reactions, or applications requiring multiple external or ported conditioning steps. The design flexibility of void chamber integration allows for implementation of sophisticated quality control measures within the integrated analytical workflow, such as dedicated staging regions associated with internal standard addition, reaction monitoring through spectroscopic measurement in an interfaced embodiment, or parallel processing for method validation, capabilities that are difficult to achieve with conventional single-function extraction devices that lack controlled staging environments. Furthermore, an embodiment of the invention enables temperature-controlled reaction staging where individual void chambers or external conditioning vessels can be subjected to selected temperatures before liquid proceeds to a downstream module.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A staged analytical processing device, comprising:
a housing defining a single continuous fluid path between a fluid inlet region and a fluid outlet region;
a plurality of discrete porous processing modules disposed sequentially within the single continuous fluid path, each discrete porous processing module comprising a porous body having interconnected pores defining internal pore surfaces,
wherein the porous body of each discrete porous processing module functions as a stationary phase such that a functional chemistry is borne on the internal pore surfaces of the porous body and analytes in a fluid sample interact with the functional chemistry during passage through the interconnected pores;
wherein at least a first discrete porous processing module bears a first functional chemistry providing a first analytical interaction mechanism and at least a second discrete porous processing module bears a second functional chemistry different from the first functional chemistry and providing a second analytical interaction mechanism different from the first analytical interaction mechanism, such that the first and second discrete porous processing modules perform different analytical operations on the fluid sample;
at least one functional staging region disposed within the single continuous fluid path between adjacent discrete porous processing modules,
the at least one functional staging region comprising a void volume configured to provide at least one of hydrodynamic buffering between adjacent discrete porous processing modules, chemical isolation of one discrete porous processing module from another, controlled timing of contact between liquid from an upstream module and chemistry of a downstream module, buffer exchange between sequential analytical operations, external conditioning of liquid before downstream module contact, or reaction staging for intermediate chemical or biochemical transformations;
wherein each discrete porous processing module is dimensioned to seal against interior walls of the housing so as to prevent bypass flow around individual discrete porous processing modules; and
a fluid interface configured to enable passage of the fluid sample through the plurality of discrete porous processing modules sequentially along the single continuous fluid path.

2. The device of claim 1, wherein the plurality of discrete porous processing modules comprises at least three discrete porous processing modules disposed sequentially within the single continuous fluid path, and wherein at least two functional staging regions are disposed between adjacent ones of the at least three discrete porous processing modules.

3. The device of claim 1, wherein the at least one functional staging region has a volume in the range of approximately 5 microliters to approximately 200 microliters, the volume being selected to provide a residence time appropriate for at least one of isolation, mixing, equilibration, externally conditioned reagent addition, port-mediated reagent addition, or incubation of an intermediate reaction.

4. The device of claim 1, wherein the fluid interface is configured for oscillatory aspirate and dispense operation such that the fluid sample is repeatedly aspirated and dispensed through the plurality of discrete porous processing modules, thereby enhancing mass transfer between the fluid sample and the functional chemistry on the internal pore surfaces of each discrete porous processing module.

5. The device of claim 1, wherein the first analytical interaction mechanism and the second analytical interaction mechanism are independently selected from the group consisting of adsorption, ion exchange, affinity binding, metal chelation, catalysis by an immobilized enzyme, and hydrophobic partitioning.

6. The device of claim 5, wherein at least one discrete porous processing module bears a functional chemistry comprising an immobilized enzyme on the internal pore surfaces of its porous body, and at least one other discrete porous processing module bears a functional chemistry comprising a chromatographic separation chemistry on the internal pore surfaces of its porous body, such that the device is configured to perform both a biochemical transformation and a chromatographic separation within the single continuous fluid path.

7. The device of claim 1, wherein at least one discrete porous processing module bears a functional chemistry selected from the group consisting of a reversed-phase chemistry, a strong cation exchange chemistry, an immobilized metal affinity chemistry, an immobilized trypsin chemistry, an antibody or antibody-fragment affinity ligand, and a mixed-mode chemistry.

8. The device of claim 1, wherein the housing is configured as a pipette tip and each discrete porous processing module is disposed within a conus region of the pipette tip.

9. The device of claim 1, wherein the porous body of at least one discrete porous processing module comprises a sintered polyolefin having internal pore surfaces that have been plasma-treated in the presence of fluorine-containing species prior to bearing the functional chemistry, the plasma treatment reducing non-specific analyte binding to the porous body while enhancing adhesion of the functional chemistry to the internal pore surfaces.

10. The device of claim 1, wherein the functional chemistry of at least one discrete porous processing module comprises a sorptive polymer coating that remains above its glass transition temperature at room temperature, the sorptive polymer coating being applied to the internal pore surfaces of the porous body such that analytes dissolve into the sorptive polymer coating by partitioning rather than by surface adsorption alone.

11. The device of claim 1, wherein the porous body of each discrete porous processing module has a pore size in the range of 10 to 200 micrometers and the functional chemistry on the internal pore surfaces has a thickness of approximately 3 micrometers, such that open flow channels are maintained through each discrete porous processing module while providing short diffusion paths between a mobile phase flowing through the interconnected pores and the functional chemistry on the internal pore surfaces.

12. The device of claim 1, wherein the functional chemistry borne by at least one discrete porous processing module is selected from the group consisting of hydrophilic interaction chemistry, hydrophobic interaction chemistry, boronate affinity chemistry, immunoaffinity chemistry, aptamer affinity chemistry, phosphopeptide enrichment chemistry, glycopeptide enrichment chemistry, derivatization chemistry, reduction chemistry, alkylation chemistry, desalting chemistry, and molecular-weight selection chemistry.

13. The device of claim 1, wherein at least one discrete porous processing module comprises a plasma-fluorinated sintered thermoplastic porous body bearing a multi-modal functional chemistry that combines a sorptive polymer mechanism with a surface-interaction mechanism.

14. The device of claim 1, wherein the plurality of discrete porous processing modules comprises a first module configured for analyte or precursor capture, a second module configured for chemical or biochemical transformation, and a third module configured for cleanup, desalting, re-capture, or chromatographic purification of products of the chemical or biochemical transformation.

15. The device of claim 1, wherein the first functional chemistry and the second functional chemistry are selected to perform chemically incompatible operations separated by the at least one functional staging region, the at least one functional staging region being configured to alter or buffer at least one of pH, ionic strength, solvent strength, reagent concentration, or incubation time between the chemically incompatible operations.

16. The device of claim 1, wherein at least one of the plurality of discrete porous processing modules is substantially free of loose particulate sorbent retained between passive frits, and wherein the functional chemistry is carried directly by the internal pore surfaces of the porous body.

17. A method for sequential multi-step sample processing within a single device, comprising:
providing a staged analytical processing device comprising a housing defining a single continuous fluid path,
a plurality of discrete porous processing modules disposed sequentially within the single continuous fluid path, and
at least one functional staging region disposed between adjacent discrete porous processing modules,
wherein each discrete porous processing module comprises a porous body having interconnected pores with internal pore surfaces bearing a functional chemistry, and
wherein the porous body of each discrete porous processing module functions as a stationary phase;
contacting a fluid sample with a first discrete porous processing module to perform a first analytical operation on the fluid sample using a first functional chemistry borne on the internal pore surfaces of the first discrete porous processing module;
transitioning processed fluid from the first discrete porous processing module through the at least one functional staging region,
wherein the at least one functional staging region provides at least one of hydrodynamic buffering, chemical isolation, controlled timing of contact with the second discrete porous processing module, external conditioning, buffer exchange, or reaction staging; and
contacting the processed fluid with a second discrete porous processing module to perform a second analytical operation different from the first analytical operation using a second functional chemistry different from the first functional chemistry, the second functional chemistry being borne on the internal pore surfaces of the second discrete porous processing module;
wherein the first analytical operation and the second analytical operation are performed sequentially within the single device without transferring the fluid sample to a separate device.

18. The method of claim 12, wherein the first analytical operation comprises capturing target analytes from a complex sample matrix on the first discrete porous processing module, and the second analytical operation comprises performing a biochemical transformation of the captured target analytes on or through the second discrete porous processing module, the method further comprising contacting products of the biochemical transformation with at least one of the first discrete porous processing module or a third discrete porous processing module to perform a third analytical operation comprising re-capture or purification of the products, thereby implementing a capture, transformation, and re-capture workflow within the single device.

19. The method of claim 12, wherein the contacting the fluid sample with the first discrete porous processing module and the contacting the processed fluid with the second discrete porous processing module each comprise oscillatory aspirating and dispensing of the fluid through the respective discrete porous processing module to enhance mass transfer between the fluid and the functional chemistry on the internal pore surfaces of the porous body.

20. The method of claim 12, wherein the first analytical operation comprises ion exchange capture of proteins from a biological sample under acidic conditions, the transitioning comprises eluting the captured proteins into the at least one functional staging region under conditions compatible with enzymatic digestion, and the second analytical operation comprises enzymatic digestion of the eluted proteins by contact with an immobilized protease borne on the internal pore surfaces of the second discrete porous processing module, the method further comprising re-capturing resulting peptides on the first discrete porous processing module for desalting and cleanup prior to analytical detection.

21. The method of claim 12, wherein the first analytical operation and the second analytical operation employ different interaction mechanisms independently selected from the group consisting of adsorption, ion exchange, affinity binding, metal chelation, catalysis by an immobilized enzyme, and hydrophobic partitioning.

22. The method of claim 12, wherein the transitioning comprises adjusting at least one of pH, ionic strength, or buffer composition of the processed fluid externally after dispensing the processed fluid from the device, or through a port communicating with the at least one functional staging region, prior to contacting the processed fluid with the second discrete porous processing module.

23. The method of claim 12, wherein the fluid sample comprises a biological fluid selected from the group consisting of blood, plasma, serum, urine, saliva, cerebrospinal fluid, and cell lysate.

24. A system for high-throughput sequential multi-step sample processing, comprising:
an automated liquid handling apparatus;
a plurality of staged analytical processing devices arranged in a multi-well format, each staged analytical processing device comprising a housing defining a single continuous fluid path,
a plurality of discrete porous processing modules disposed sequentially within the single continuous fluid path, and
at least one functional staging region disposed between adjacent discrete porous processing modules,
wherein each discrete porous processing module comprises a porous body having interconnected pores with internal pore surfaces bearing a functional chemistry, and
wherein the porous body of each discrete porous processing module functions as a stationary phase;
a multi-well collection vessel configured to receive processed fluid from each of the plurality of staged analytical processing devices; and
wherein the automated liquid handling apparatus is configured to perform oscillatory aspirate and dispense cycles through the plurality of staged analytical processing devices to sequentially process a plurality of fluid samples through the discrete porous processing modules of each staged analytical processing device.

25. The system of claim 19, wherein at least a first subset of the plurality of staged analytical processing devices is configured with a first combination of discrete porous processing modules providing a first analytical workflow, and at least a second subset of the plurality of staged analytical processing devices is configured with a second combination of discrete porous processing modules providing a second analytical workflow different from the first analytical workflow, thereby enabling parallel processing of different analytical protocols across the multi-well format.
